SEQUENCE LISTING (35 sequences)

SEQ ID NO: 1 - protein, 20 aa, Artificial Sequence
  Synthetic Peptide
KGGGQKCIVQTTSWSQCSKS

SEQ ID NO: 2 - protein, 19 aa, Artificial Sequence
  Synthetic Peptide
GGGQKCIVQTTSWSQCSKS

SEQ ID NO: 3 - protein, 13 aa, Artificial Sequence
  Synthetic Peptide
KYGLALERKDHSG

SEQ ID NO: 4 - protein, 12 aa, Artificial Sequence
  Synthetic Peptide
YGLALERKDHSG

SEQ ID NO: 5 - protein, 23 aa, Artificial Sequence
  Snythetic Peptide
KGGSINNNRWHSIYITRFGNMGS

SEQ ID NO: 6 - protein, 22 aa, Artificial Sequence
  Synthetic Peptide
GGSINNNRWHSIYITRFGNMGS

SEQ ID NO: 7 - protein, 15 aa, Artificial Sequence
  Snythetic Peptide
KGGTWYKIAFQRNRK

SEQ ID NO: 8 - protein, 14 aa, Artificial Sequence
  Snythetic Peptide
GGTWYKIAFQRNRK

SEQ ID NO: 9 - protein, 15 aa, Artificial Sequence
  Snythetic Peptide
KGGTSIKIRGTYSER

SEQ ID NO: 10 - protein, 14 aa, Artificial Sequence
  Snythetic Peptide
GGTSIKIRGTYSER

SEQ ID NO: 11 - protein, 16 aa, Artificial Sequence
  Snythetic Peptide
KYGTDIRVTLNRLNTF

SEQ ID NO: 12 - protein, 15 aa, Artificial Sequence
  Snythetic Peptide
YGTDIRVTLNRLNTF

SEQ ID NO: 13 - protein, 16 aa, Artificial Sequence
  Snythetic Peptide
KYGSETTVKYIFRLHE

SEQ ID NO: 14 - protein, 15 aa, Artificial Sequence
  Snythetic Peptide
YGSETTVKYIFRLHE

SEQ ID NO: 15 - protein, 15 aa, Artificial Sequence
  Snythetic Peptide
KYGKAFDITYVRLKF

SEQ ID NO: 16 - protein, 14 aa, Artificial Sequence
  Snythetic Peptide
YGKAFDITYVRLKF

SEQ ID NO: 17 - protein, 15 aa, Artificial Sequence
  Snythetic Peptide
KYGAASIKVAVSADR

SEQ ID NO: 18 - protein, 14 aa, Artificial Sequence
  Snythetic Peptide
YGAASIKVAVSADR

SEQ ID NO: 19 - protein, 15 aa, Artificial Sequence
  Snythetic Peptide
KGGNGEPRGDTYRAY

SEQ ID NO: 20 - protein, 14 aa, Artificial Sequence
  Snythetic Peptide
GGNGEPRGDTYRAY

SEQ ID NO: 21 - protein, 14 aa, Artificial Sequence
  Snythetic Peptide
CGGNGEPRGDTRAY

SEQ ID NO: 22 - protein, 15 aa, Artificial Sequence
  Snythetic Peptide
KYGRKRLQVQLSIRT

SEQ ID NO: 23 - protein, 14 aa, Artificial Sequence
  Snythetic Peptide
YGRKRLQVQLSIRT

SEQ ID NO: 24 - protein, 13 aa, Artificial Sequence
  Snythetic Peptide
KGGRNIAEIIKDI

SEQ ID NO: 25 - protein, 12 aa, Artificial Sequence
  Snythetic Peptide
GGRNIAEIIKDI

SEQ ID NO: 26 - protein, 15 aa, Artificial Sequence
  Snythetic Peptide
KGGPQVTRGDVFTMP

SEQ ID NO: 27 - protein, 14 aa, Artificial Sequence
  Snythetic Peptide
GGPQVTRGDVFTMP

SEQ ID NO: 28 - protein, 15 aa, Artificial Sequence
  Snythetic Peptide
GGPQVTRGDVFTMPK

SEQ ID NO: 29 - protein, 7 aa, Artificial Sequence
  Snythetic Peptide
GRGDSPK

SEQ ID NO: 30 - protein, 15 aa, Artificial Sequence
  Snythetic Peptide
KGGAVTGRGDSPASS

SEQ ID NO: 31 - protein, 14 aa, Artificial Sequence
  Snythetic Peptide
GGAVTGRGDSPASS

SEQ ID NO: 32 - protein, 16 aa, Artificial Sequence
  Snythetic Peptide
YAALPQVTRGNVFTMP

SEQ ID NO: 33 - protein, 5 aa, Artificial Sequence
  Snythetic Peptide
RGDYK

SEQ ID NO: 34 - protein, 12 aa, Artificial Sequence
  Snythetic Peptide
GGVTRGNVFTMP

SEQ ID NO: 35 - protein, 13 aa, Artificial Sequence
  Snythetic Peptide
GGNGEPRGDTRAY

(19) United States
(12) Patent Application Publication
(10) Pub. No.: US 2011/0183418 A1
(43) Pub. Date: Jul. 28, 2011

(54) Peptide-Polymer Cell Culture Articles and Methods of Making

(76) Inventors: Arthur Winston Martin, Horseheads, NY (US); Jodelle Karen Nelson, Corning, NY (US); Christopher Bankole Shogbon, Corning, NY (US); David Michael Weber, Big Flats, NY (US); Yue Zhou, Horseheads, NY (US)

(21) Appl. No.: 12/845,411

(22) Filed: Jul. 28, 2010

Related U.S. Application Data
(60) Provisional application No. 61/229,615, filed on Jul. 29, 2009.

(51) Int. Cl. C12N 5/071; C07K 14/00; C07K 7/08; C07K 7/06; C08H 1/00; C07K 2/00

(52) U.S. Cl. 435/396; 530/326; 530/327; 530/329; 530/330; 526/238.1; 530/317

(57) ABSTRACT

Functionalized peptide monomers, peptides that have been functionalized to contain a polymerization moiety, are disclosed. The use of these functionalized peptide monomers to form peptide polymers which are useful as synthetic surfaces capable of supporting culture of cells in culture, particularly cells that will be used therapeutically, is also disclosed. Methods of making the surfaces and methods of using the surfaces are also disclosed.

### CLAIMING BENEFIT OF PRIOR FILED U.S. APPLICATION

This application claims the benefit of U.S. Provisional Application Ser. No. 61/229,615, filed on Jul. 29, 2009. The content of this document and the entire disclosure of publications, patents, and patent documents mentioned herein are incorporated by reference.

### FIELD

The present disclosure relates to peptide-polymer cell culture surfaces and articles and methods for preparing peptide-polymer cell culture articles via polymerization of functionalized cell binding peptides with synthetic monomers. More particularly, the disclosure relates to synthetic surfaces having cell binding peptides and articles for supporting the culture of cells, including undifferentiated stem cells in chemically defined medium.

### SEQUENCE LISTING

This application contains a Sequence Listing electronically submitted via EFS-Web to the United States Patent and Trademark Office as text filed named “2010722_SP09-224_ST25.txt” having a size of 8 kb and created on Jul. 7, 2010. Due to the electronic filing of the Sequence Listing, the electronically submitted Sequence Listing serves as both the paper copy required by 37 CFR §1.821(c) and the CRF required by §1.821(e). The information contained in the Sequence Listing is hereby incorporated herein by reference.

### BACKGROUND

Therapeutic cells, cells which may be introduced into a human for the treatment of disease, are being developed. Examples of therapeutic cells include pluripotent stem cells such as human embryonic stem cells (hESCs) which have the ability to differentiate into any of the three germ layers, giving rise to any adult cell type in human body. This property of stem cells provides a potential for developing new treatments for a number of serious cell degenerative diseases, such as diabetes, spinal chord injury, heart diseases and the like. However there remain obstacles in the development of such therapeutic cell-based treatments. Obtaining and maintaining adequate numbers of therapeutic cells in cell and tissue culture and ensuring that these cells do not change in unwanted ways during cell culture are important in developing and controlling therapeutic cell cultures. For example, stem cell cultures, such as hESC cell cultures are typically seeded with a small number of cells from a cell bank or stock and then amplified in the undifferentiated state until differentiation is desired for a given therapeutic application. To accomplish this, the hESC or their differentiated cells are currently cultured in the presence of surfaces or media containing animal-derived components, such as feeder layers, serum, or Matrigel™ available from BD, Franklin Lakes, N.J. These animal-derived additions to the culture environment expose the cells to potentially harmful viruses or other infectious agents which could be transferred to patients or compromise general culture and maintenance of the hESCs. In addition, such biological products are vulnerable to batch variation, immune response and limited shelf-life.

### SUMMARY

In embodiments of the present invention, a functionalized peptide monomer is disclosed, where the peptide has been functionalized to contain a polymerization moiety such as a (meth)acrylate moiety. In embodiments, the functionalized peptide monomer can be described by the formula: Aₘ-S_{y}-Xaaₙ-S_{y1}-Z-S_{y2}-Aₘ₁ where A is a polymerization moiety, m is an integer from 1 to 6, m1 is an integer from 1 to 6, Xaaₙ is independently any amino acid, n is an integer from 0 to 20, S is a spacer, y is an integer from 0 to 30, y1 is an integer from 0 to 30, y2 is an integer from 0 to 30, and Z is a cell adhesive peptide. In embodiments, the cell adhesive peptide may be a cell adhesive peptide, and may be a cell adhesive peptide having an RGD sequence, for example, KGGGQKCIVQTTSWSQCSKS (SEQ ID NO: 1); GGGQKCIVQTTSWSQCSKS(SEQ ID NO:2); KYGLALERKDHSG (SEQ ID NO:3); YGLALERKDHSG (SEQ ID NO:4); KGGSINNNRWHSIYITRFGNMGS (SEQ ID NO:5); GGSINNNRWHSITYITRFGNMGS (SEQ ID NO:6); KGGTWYKIAFQRNRK (SEQ ID NO:7); GGTWYKIAFQRNRK (SEQ ID NO:8); KGGTSIKIRGTYSER (SEQ ID NO:9); GGTSIKIRGTYSER (SEQ ID NO:10); KYGTDIRVTLNRLNTF (SEQ ID NO:11); YGTDIRVTLNRLNTF (SEQ ID NO:12); KYGSETTVKYLFRLHE (SEQ ID NO:13); YGSETTVKYIFRLHE(SEQ ID NO:14); KYGKAFDITYVRLKF (SEQ ID NO:15); YGKAFDITYVRLKF(SEQ ID NO:16); KYGAASIKVAVSADR (SEQ ID NO:17); YGAASIKVAVSADR(SEQ ID NO:18); KGGNGEPRGDTYRAY(SEQ ID NO:19); GGNGEPRGDTYRAY (SEQ ID NO:20) CGGNGEPRGDTYRAY (SEQ ID NO:21); GGNGEPRGDTRAY (SEQ ID NO:22); KYGRKRLQVQLSIRT (SEQ ID NO:23); YGRKRLQVQLSIRT(SEQ ID NO:24); KGGRNIAEIIKDI (SEQ ID NO:25); GGRNIAEIIKDI (SEQ ID NO:26); KGGPQVTRGDVFTMP (SEQ ID NO:27); GGPQVTRGDVFTMP(SEQ ID NO:28); GGPQVTRGDVFTMPK (SEQ ID NO:29); GRGDSPK (SEQ ID NO:30); KGGAVTGRGDSPASS(SEQ ID NO:31); GGAVTGRGDSPASS (SEQ ID NO:32); Yaa₁PQVTRGNVFTMP (SEQ ID NO:32); RGDYK (SEQ ID NO:34), where RGDYK (SEQ ID NO: 34) may be cyclic, or combinations.

In embodiments, the adhesive peptide of the functionalized peptide monomer comprises KGGPQVTRGDVFTMP (SEQ ID NO:27) or GGPQVTRGDVFTMP (SEQ ID NO:28), GGNGEPRGDTYRAY (SEQ ID NO:18) or KGGNGEPRGDTYRAY (SEQ ID NO:19). In embodiments, the photopolymerizable moiety comprises an acrylate or a methacrylate, for example, methacrylic acid. In embodiments, the spacer is polyethylene oxide which may be, for example PEG₄.

In additional embodiments the invention provides methods of making a cell culture surface comprising the steps of: providing a mixture of functionalized peptide monomer and at least one (meth)acrylate monomer; applying the mixture to a cell culture substrate; and, polymerizing the monomers to form a peptide-polymer. In embodiments, the (meth)acrylate monomer comprises HEMA, TEGDMA, Glycerol monomethacrylate or glycerol 1,3-diglycerolate dimethacrylate or combinations. In embodiments, the methacrylate monomer comprises a combination of HEMA and TEGDA.

In additional embodiments of the present invention, a method is described for culturing an isolated population of undifferentiated human embryonic stem cells in chemically defined medium on the peptide-polymer culture surface.

### BRIEF DESCRIPTION OF THE DRAWINGS

FIG. 1 is a flow chart showing an embodiment of a method of making cell culture surfaces according to embodiments.

FIG. 2 is a schematic illustration showing a method for making an embodiment of a cell culture surface.

FIG. 3A-B shows the fluorescence intensity of fluorescently labeled peptide polymer distributed over a cell culture surface.

FIG. 4A-C shows photomicrographs of H7 human embryonic stem cells cultured on Matrigel™ (MG, FIG. 4A), Synthemax™ (FIG. 4B) control surfaces and on a functionalized peptide-grafted surface embodiment of the present invention (FIG. 4C).

FIG. 5A-F shows photomicrographs of H7 crystal violet-stained human embryonic stem cells cultured on Matrigel™ (MG, FIG. 5A), Synthemax™ (FIG. 5B) control surfaces, and two embodiments of the functionalized peptide-grafted surfaces (C,D) and (E,F). FIGS. 5C and D show the same conditions. FIGS. 5E and F show the same conditions.

FIG. 6A-H show photomicrographs of neuronal progenitor cells growing on Laminin™ surface from Corning, Incorporated (FIGS. 6A and E), on a cyclic functionalized RGD peptide surface made with HEMA and TEGDMA (FIGS. 6B and F), on a cyclic functionalized RGD peptide surface made with glycerol methacrylate and TEGDMA (FIGS. 6C and G) and on a Synthemax™ surface from Corning Incorporated (FIGS. 6D and H).

### DETAILED DESCRIPTION

In embodiments of the present invention, a functionalized peptide monomer is disclosed, where the peptide has been functionalized to contain a polymerization moiety such as a (meth)acrylate moiety. In embodiments, the functionalized peptide monomer can be described by the formula: Aₘ-S_{y}-Xaaₙ-S_{y1}-Z-S_{y2}-Aₘ₁ where A is a polymerization moiety, m is an integer from 1 to 6. m1 is an integer from 0 to 6, Xaaₙ is independently any amino acid, n is an integer from 0 to 6, S is a spacer, y is an integer from 0 to 30, y1 is an integer from 0 to 30, y2 is an integer from 0 to 30, and Z is a cell adhesive peptide. In embodiments, methods of using the functionalized peptide monomer to form a polymeric cell culture surface incorporating and methods of using the polymeric cell culture surface so made are also disclosed. In embodiments, a polymerization moiety is present at or near both the carboxyl end and the amino end of the peptide.

In the field of cell culture, culturing cells in a scalable fashion requires surfaces that are free of pathogens, relatively inexpensive, stable and reliable, and support long term culture of cells in culture. This is particularly true for cell culture aimed at providing therapeutic cells. That is, cell culture aimed at providing cells which will be introduced or re-introduced into a human for the treatment of disease. While current technology for cell culture includes surfaces that are derived from animal products such as Matrigel™, available from BD, Franklin Lakes N.J., derived from mouse tumor extract, these surfaces are not desirable for support of cells that will be used therapeutically.

Embodiments of the present invention provide peptide mimetic cell culture surfaces or coatings prepared by free radical polymerization of acrylate or methacrylate functionalized cell adhesive peptides with hydrophilic acrylate or methacrylate monomers and cross-linkers. In embodiments, also provided are methods to create a peptide mimetic surface that is scalable, easy to process, cost effective and can facilitate the growth and proliferation of difficult to culture cells such as human Embryonic Stem Cells (hESCs) for therapeutic value on a larger surface area such as T-75 Flask and T-225 Flask. In embodiments, the invention provides a facile, cost effective, and manufacturing friendly process for producing peptide mimetic surfaces. In embodiments, these surfaces are loosely cross-linked, hydrophilic polymeric surfaces that are chemically linked to adhesive peptides and can be coated on large foot-prints of thermoplastic surfaces for producing the quantity of cells required for therapeutic uses.

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to.” Synthetic cell culture surfaces including surfaces that incorporate synthetic or recombinant proteins or peptides, for example, are suitable for supporting cells that may be introduced into a human for the treatment of disease. Synthetic peptides and proteins often include cell adhesive sequences such as RGD. In embodiments, the cell adhesive sequence may be an integrin binding sequence of extracellular matrix origin.

Some exemplary synthetic peptides and proteins that include RGD sequences have been identified from proteins such as Laminins, vitronectin, bone sialoprotein (BSP) and others. Some of these sequences are shown in Table 1. Synthetic surfaces that reduce the amount of peptide required to support viable cells in culture are desirable, because peptides can expensive, and so surfaces requiring less peptide are less expensive. In addition, synthetic surfaces that are easy to manufacture, stable in storage, stable through sterilization procedures, and stable through long exposure to aqueous cell culture conditions are also desirable.

In embodiments, the functionalized peptide has the following formula (Formula 1):

Formula 1: Aₘ-S_{y}-Xaaₙ-S_{y1}-Z-S_{y2}Aₘ₁

In embodiments, peptides or polypeptides (Z) which have been modified or functionalized to carry at least two polymerizable moieties (A), where A is an α, β unsaturated group or ethylenically unsaturated group which includes, for example, an acrylate, methacrylate, acrylamide, methacrylamide, maleimide or fumarate, one such polymerizable group at or near each end, the carboxyl end and the amino end, of the functionalized peptide, are provided. For the purposes of this disclosure, “at or near” means that the polymerizable moiety (A) may be adjacent to the peptide sequence, or separated from the peptide sequence by one or more spacers, where the spacers may be polyethylene oxide spacers or amino acid spacers or a combination. For the purposes of this disclosure “functionalized peptide” means peptides which have been modified to incorporate at least two polymerization moieties or polymerizable moieties such as acrylate, methacrylate, acrylamide, methacryalmide, maleimide or fumarate groups, and optionally spacers (S) and/or additional amino acid sequences (Xaa). Epoxide functional groups may also be polymerizable moieties.

In embodiments these polymerization moieties can form polymers upon exposure to an energy source. In embodiments, photopolymerization moieties are bound to the functionalized peptide through the side chains of amino acids. For example, a methacrylate, acrylamide, and maleimide moiety may be bound to the side chain of a lysine amino acid (Xaa-MAA) where Xaa is lysine and MAA is methacrylic acid) but can be any other carboxyl functionalized acrylate, methacrylate and acrylamide, to create a functionalized peptide. The polymerization moiety may be bound to the sidechain of an amino acid in the cell adhesive peptide, or in the Xaaₙ group.

As shown in Formula 1, where A is independently a polymerization moiety, m, in embodiments, is an integer from 1 to 6 and m1 is an integer from 1 to 6. In embodiments, functionalized peptides may optionally also comprise spacers. In embodiments the polymerization moiety (Aₘ and Aₘ₁) may be bound to the side chain of an amino acid (for example K or R) or may be bound to the N-terminus of an amino acid or peptide. The polymerization moiety may attach to the spacer, Sₚ through the polyethylene oxide, through the side chain of an amino acid such as lysine or at the N-terminus of the amino acid.

In embodiments, (S_{y}, S_{y1} and S_{y2}) may be a polyalkylene oxide including for example polyethylene glycol (PEG) or polypropylene glycol (PPG) which are represented by the formula (O—CH₂CHR′)ₘ₂ where R′ is H or CH₃ and m2 is an integer from 0 to 20. In embodiments, relatively short chains of polyalkylene oxide are desirable. For example, in embodiments, y, y1 and y2 may be PEG₂, PEG₄, PEG₆, PEG₈, PEG₁₀, PEG₁₂ or PPG₂, PPG₄, PPG₆, PPG₈, PPG₁₀, PPG₁₂ or PPG₂₀. In embodiments, the spacer is a polyethylene oxide with 20 or fewer repeating units (i.e. PEG₄, PEG₆, PEG_{S}, PEG₁₀, PEG₁₂, PEG₁₄, PEG₁₆, PEG₁₈ or PEG₂₀).

In embodiments S_{y}, S_{y1} and/or S_{y2} are PPG or PEG having a functional group. For example, the PEG or PPG spacer may have a maleimide, thiol, amine, silane, aldehyde, epoxide, isocyanate, acrylate or carboxyl group. In embodiments the PEG spacer is a Jeffamine, a PEG having an amine functional group. In additional embodiments, the PEG or PPG may be branched. For example the branched PEG or PPO may be a Y-branched or star-PEG or PPG. In embodiments these branched PEG or PPO spacers may allow multiple peptides to be conjugated to a base material through a single functional peptide.

Xaa is independently any amino acid which may be present or absent, or a group of up to thirty amino acids (where n is an integer from 0 to 30), up to twenty amino acids (where n is from 0 to 20), up to ten amino acids (where n is an integer from 0 to 10), up to six amino acids (where n is an integer from 0 to 6), or up to three amino acids (where n is an integer from 0 to 3). In embodiments, the Xaa amino acid contains a Lysine amino acid which can bind a photopolymerizable group. In embodiments the Xaa amino acid sequence may be a sequence as, for example, LysGlyGly or LysTyrGly. The term “independently” is used herein to indicate that each aa may differ from another amino acid. In embodiments, Xaa is acetylated. Amino acid Xaaₙ may be acetylated and/or amidated to protect it from degradation. In embodiments Xaa may be a hydrophilic amino acid. For example, Xaaₙ may comprise a lysine, glysine, glutamic acid, serine, aspartic acid or arginine amino acid, which may be a terminal amino acid. For example, in embodiments, Xaaₙ may be an amino acid Xaaₙ where Xaa is G and where n=1 to 20, or Xaaₙ may be an amino acid where Xaa is K and n=1 to 20 or Xaa is K and n=n is greater than or equal to 1, or Xaaₙ may be an amino acid Xaaₙ where Xaa is D and n=1 to 20, or Xaaₙ may be an amino acid Xaaₙ where Xaa is E and n=1 to 20. For example, the functionalized peptide may be MAA-Lys-Lys-Lys-Lys-Lys-Lys-Lys-VN-Peptide (n-terminal attachment to lysine alpha terminal) or Ac-(Lys-Lys-Lys-Lys-Lys-Lys-MAA)-VN-Peptide (Methacrylate linked) to a series of lysine spacer length sprung from an epsilon lysine side chain. The MAA can be attached on the n-terminal of the spacer length or it can be formed on a lysine side chain. In embodiment, Xaaₙ may be a three amino acid sequence such as LysGlyGly or LysTyrGly. In embodiments, Xaaₙ is a series of the same amino acid. Xaaₙ may comprise a hydrophilic amino acid such as lysine, glycine, glutamic acid, aspartic acid or arginine amino acid. In embodiments, Xaaₙ may have a terminal lysine or arginine.

The peptide Z may be a cell adhesive peptide. For the purposes of this disclosure a cell adhesive peptide is a peptide or polypeptide, an amino acid sequence (which terms are interchangeable) which contains a sequence known or after-identified to enhance cell binding, either to a surface in cell culture, or to other cells or extracellular matrix in vivo. Such as a peptide may contain an RGD sequence. In addition, the peptide may be acetylated or amidated to provide stability and protect the small amino acid sequence from cleavage. For the purposes of this disclosure, peptide or polypeptide is an amino acid sequence that may be chemically synthesized or made by recombinant methods. However, for the purposes of this disclosure, peptide or polypeptide is a fragment of a protein, and not a complete protein. In addition, in embodiments, peptide or polypeptide is not isolated from an animal source. In embodiments, peptide or polypeptide may include an amino acid sequence of Yaa₁ProGlnValThrArgGlyAspValPheThrMetPro (SEQ ID NO:33), a vitronectin peptide sequence where 1 is an integer from 0 to 3 and where Yaa may be any amino acid or may be, for example, lysine.

Examples of peptides (Z) that may be used in embodiments are listed in Table 1. However, any suitable peptide sequence may be used.

TABLE 1

| Sequence | Source |
|---|---|
| KGGGQKCIVQTTSWSQCSKS | Cyr61 res 224-240 |
| (SEQ ID NO: 1) | |
| GGGQKCIVQTTSWSQCSKS | Cyr61 res 224-240 |
| (SEQ ID NO: 2) | |
| KYGLALERKDHSG | TSP1 res 87-96 |
| (SEQ ID NO: 3) | |
| YGLALERKDHSG | TSP1 res 87-96 |
| (SEQ ID NO: 4) | |
| KGGSINNNRWHSIYITRFGNMGS | mLMα1 res 2179-2198 |
| (SEQ ID NO: 5) | |
| GGSINNNRWHSIYITRFGNMGS | mLMα1 res 2179-2198 |
| (SEQ ID NO: 6) | |
| KGGTWYKIAFQRNRK | mLMα1 res 2370-2381 |
| (SEQ ID NO: 7) | |
| GGTWYKIAFQRNRK | mLMα1 res 2370-2381 |
| (SEQ ID NO: 8) | |
| KGGTSIKIRGTYSER | mLMγ1 res 650-261 |
| (SEQ ID NO: 9) | |
| GGTSIKIRGTYSER | mLMγ1 res 650-261 |
| (SEQ ID NO: 10) | |
| KYGTDIRVTLNRLNTF | mLMγ1 res 245-257 |
| (SEQ ID NO: 11) | |
| YGTDIRVTLNRLNTF | mLMγ1 res 245-257 |
| (SEQ ID NO: 12) | |
| KYGSETTVKYIFRLHE | mLMγ1 res 615-627 |
| (SEQ ID NO: 13) | |
| YGSETTVKYIFRLHE | mLMγ1 res 615-627 |
| (SEQ ID NO: 14) | |
| KYGKAFDITYVRLKF | mLMγ1 res 139-150 |
| (SEQ ID NO: 15) | |
| YGKAFDITYVRLKF | mLMγ1 res 139-150 |
| (SEQ ID NO: 16) | |
| KYGAASIKVAVSADR | mLMα1 res 2122-2132 |
| (SEQ ID NO: 17) | |
| YGAASIKVAVSADR | mLMα1 res 2122-2132 |
| (SEQ ID NO: 18) | |
| KGGNGEPRGDTYRAY | BSP |
| (SEQ ID NO: 19) | |
| GGNGEPRGDTYRAY | BSP |
| (SEQ ID NO: 20) | |
| CGGNGEPRGDTRAY | BSP-Y |
| (SEQ ID NO: 21) | |
| GGNGEPRGDTRAY | BSP-Y |
| (SEQ ID NO: 22) | |
| KYGRKRLQVQLSIRT | mLMαl res 2719-2730 |
| (SEQ ID NO: 23) | |
| YGRKRLQVQLSIRT | mLMαl res 2719-2730 |
| (SEQ ID NO: 24) | |
| KGGRNIAEIIKDI | LMβ1 |
| (SEQ ID NO: 25) | |
| GGRNIAEIIKDI | LMβ1 |
| (SEQ ID NO: 26) | |
| KGGPQVTRGDVFTMP | VN |
| (SEQ ID NO: 27) | |
| GGPQVTRGDVFTMP | VN |
| (SEQ ID NO: 28) | |
| GGPQVTRGDVFTMPK | VN |
| SEQ ID NO: 29) | |
| GRGDSPK | Short FN |
| (SEQ ID NO: 30) | |
| KGGAVTGRGDSPASS | Long FN |
| (SEQ ID NO: 31) | |
| GGAVTGRGDSPASS | Long FN |
| (SEQ ID NO: 32) | |
| YaaₗPQVTRGNVFTMP | VN |
| (SEQ ID NO: 33) | |
| RGDYK | RGD |
| (SEQ ID NO: 34) | |
| GGVTRGNVFTMP | |
| (SEQ ID NO: 35) | |

For the purposes of this disclosure, “cyclic functionalized peptide” means a cyclic peptide sequence which is also modified to comprise a polymerization moiety and optionally spacers in the form of low molecular weight polyalkylene glycol moieties or amino acid sequences. An example of a cyclic functionalized peptide is, for example, RGDYK(SEQ ID NO:34) which, in its cyclic form can be written as c(RGDyK) (SEQ ID NO: 34) and This cyclic peptide is shown in the following formula (Formula 2):

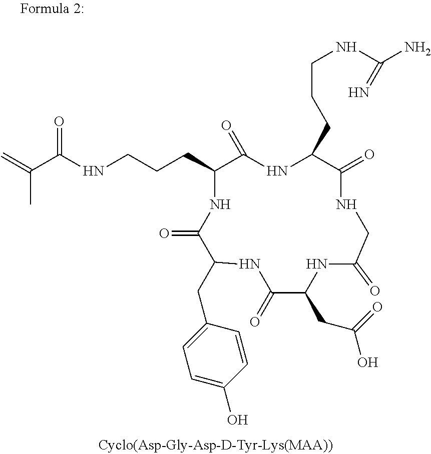

For the purposes of this disclosure “Cyclic functionalized peptides” can be illustrated according to the following formula (Formula 3):

Z_{c}-S_{y}-Xaaₙ-Aₘ Formula 3

In embodiments, according to Formula 3, Z_{c} is a cyclic peptide and S_{y}Xaaₙ and Aₘ are defined above. For example, the functionalized peptide may be a cyclic functionalized peptide with the sequence RGDYK (SEQ ID NO: 34) in cyclic form with the formula shown in Formula 2, where A is a methacrylate moiety attached to the Lysine (K) amino acid through the Lysine sidechain, m=1, y=0 and n=0.

These cyclic functionalized peptides may also optionally have a spacer moiety between the peptide and the polymerization moiety. The spacer (S_{y}) may be, for example, polyethylene oxide (PEO), polyethylene glycol (PEG) or polypropylene oxide (PPO) having repeating units where y=0 to 20.

In embodiments of the cyclic functional peptides, S_{y} may be a polyalkylene oxide including for example polyethylene glycol (PEG) or polypropylene glycol (PPG) which are represented by the formula (O—CH₂CHR′)ₘ₂ where R′ is H or CH₃ and m2 is an integer from 0 to 20. In embodiments, relatively short chains of polyalkylene oxide are desirable. For example, in embodiments, S_{y} may be PEG₂, PEG₄, PEG₆, PEG_{S}, PEG₁₀, PEG₁₂ or PPG₂, PPG₄, PPG₆, PPG₈, PPG₁₀, PPG₁₂ or PPG₂₀. Or, in embodiments, the spacer S may comprise polyethylene oxide spacer and amino acid spacer in any combination. In embodiments, S may be a hydrophobic spacer such as palmitic acid, stearic acid, lauric acid or hexaethylene diamine. In embodiments, S may be carboxyethyl methacrylate.

Others have disclosed the use of (meth)acrylic acid derivatives chemically modified with a protein or peptide for the preparation of cell culture surfaces (U.S. Pat. No. 5,643,561, the '561 patent). In the '561 patent, no disclosure is made of the use of a spacer between the peptide sequence and the polymerization moiety. Others have disclosed the use of a long chain PEG spacer, combined with a cell adhesive peptide sequence and a polymerization moiety. For example Hern, D. L., and Hubbell, J. A., Incorporation of Adhesion Peptides into Nonadhesive Hydrogels Useful for Tissue Resurfacing, Journal of Biomedical Materials Research Part A Vol. 39, Issue 2, pp. 266-276 (Hern & Hubbell) discloses the use of cell adhesive peptides conjugated to a polymerization moiety, and the use of cell adhesive peptides conjugated to polymerization moiety via a long chain polyalkylene oxide spacer (PEG75) which was combined with PEG diacrylate (copolymerized with PEG diacrylate) to form a hydrogel cell culture surface composed primarily of PEG. However, Hern and Hubbell disclose that the use of a cell adhesive peptide conjugated directly to a polymerization moiety produced a cell culture surface that did not support the specific binding of cells to the cell binding protein sequences provided. That is, cells seeded on hydrogels containing peptide incorporated via a linkage lacking a PEG spacer arm adhered nonspecifically, i.e. in a manner that required serum proteins and did not depend on the precise identity of the peptide provided. This was not desirable, according to Hern&Hubble. The use of a long chain PEG spacer (MW3400 PEG), in combination with a predominantly PEG hydrogel polymeric material supported specific cell binding. Hern & Hubbell is silent as to the use of short chain PEG spacers, such as (O—CH₂CHR′)ₘ₂ where R′ is H or CH₃ and m2 is an integer from 0 to 20. In addition, the Hern & Hubbell disclosure relates to surfaces that are primarily composed of PEG. (see also U.S. Pat. No. 7,615,593 which discloses the use of bifunctional PEG of the formula ((CH₂)m-O)ₙ where m is an integer from 2 to 8 and n is an integer greater than 100, and preferably 2,000 (column 5, line 64 to column 6, line 2).

The surfaces disclosed herein have, in embodiments, glycerol methacrylate and/or HEMA hydrophilic base substrates to which functionalized peptides are bound. HEMA and glycerol methacrylate have different cell culture characteristics compared to PEG. PEG is a non-binding surface. That is, proteins do not adsorb to PEG, and cells to not bind to PEG surfaces. PEG, in general, has a contact angle of less than 20 degrees. HEMA and glycerol methacrylate are not as non-binding as PEG, and the contact angles of surfaces prepared according to embodiments disclosed herein have contact angles of between 36 degrees and 56 degrees. While not wishing to be bound by theory, it may be that these differences in overall composition allow the functionalized peptides disclosed herein to provide functional cell culture surfaces with specific cell binding characteristics, where the primarily PEG surfaces having peptides without a PEG spacer did not provide suitable cell culture surfaces as disclosed in Hern & Hubbell.

In addition, the existence of a polymerizable moiety (other than an amino moiety inherent to a polypeptide) at or near both the carboxyl end and the amino end of the peptide has not been disclosed. Functionalized peptide or polypeptide may be provided to the substrate at any density, preferably at a density suitable to support culture of cells. Functionalized peptide may be provided to substrate at a concentration between about 0.25 mM to 2.0 mM which gives a peptide density of approximately 2 pmol to 50 pmol per mm² on and within the polymer-peptide matrix which can be estimated by the surface area of substrate that is coated in embodiments. For example, the functionalized peptide may be present at a density of greater 0.25 pmol/mm², greater than than 0.5 pmol/mm², greater than 1 pmol/mm², greater than 5 pmol/mm², greater than 6 pmol/mm², greater than 7 pmol/mm², greater than 8 pmol/mm², greater than 9 pmol/mm², greater than 10 pmol/mm², greater than 12 pmol/mm², greater than 15 pmol/mm², or greater than 20 pmol/mm², or greater than 40 pmol/mm² within the polymer-peptide loosely cross-linked network. It will be understood that the amount of peptide present can vary depending on the composition of the polymer-peptide layer, the thickness of the polymer-peptide layer and the nature of the polypeptide itself. As discussed below in the Examples, higher densities of peptide may be better able to support attachment and proliferation of undifferentiated stem cells in a chemically defined medium.

Embodiments of functionalized peptides of the present invention are described in Table 2.

TABLE 2

| | Methacrylate |
|---|---|
| | Functionalized |
| Design | Peptides |
| Linear Methacryl | Ac-Lys[Methacryl]- |
| RGD Peptide | Gly-Gly-Pro-Gln- |
| Vitronectin | Val-Thr-Arg-Gly-Asp- |
| Sequence | Val-Phe-Thr-Met-Pro- |
| | NH2 or [Methacryl]- |
| | Lys-Gly-Gly-Pro-Gln- |
| | Val-Thr-Arg-Gly-Asp- |
| | Val-Phe-Thr-Met-Pro- |
| | NH2 (Ac-Lys-MAA- |
| | SEQ ID NO: 27-NH₂) |
| Bi-functional | Ac-Lys[Methacryl]- |
| Methacryl | Gly-Gly-Pro-Gln-Val- |
| RGD Peptide | Thr-Arg-Gly-Asp-Val- |
| (Lysine C-terminal | Phe-Thr-Met-Pro-Lys- |
| attachment) of | [Methacryl] |
| second Methacrylate) | |
| Function as a | (Ac-Lys-MAA- |
| crosslinker and | SEQ ID NO: 29-MAA) |
| adhesive ligand | |
| at the same time | |
| Vitronectin Sequence | |
| Bi-functional | Ac-Lys[Methacryl]- |
| Methacryl RGD | Gly-Gly-Pro-Gln-Val- |
| Peptide (C-Linker | Thr-Arg-Gly-Asp-Val- |
| attachment) of second | Phe-Thr-Met-Pro-NH2- |
| Methacrylate | [Methacryl] (AC-Lys- |
| Vitronectin Sequence | MAA-SEQ ID NO: 27- |
| | NH₂-MAA) |
| Bi-functional | [Methacryl]-Lys-Gly- |
| Methacryl RGD | Gly-Asn-Gly-Glu-Pro- |
| Peptide (C-Linker | Arg-Gly-Asp-Thr-Tyr- |
| attachment) of second | Arg-Ala-Tyr-NH2- |
| methacrylate) | [Methacryl] |
| Function as a | (MAA-SEQ ID NO: |
| crosslinker and | 19-NH₂-MAA) |
| adhesive ligand | |
| at the same time | |
| Bone Sialoprotein | |
| (BSP) | |
| Methacrylate PEG | [Methacryl]-PEO4- |
| Functionalized Peptide | Lys-Gly-Gly-Pro-Gln- |
| (PEG spacer linker for | Val-Thr-Arg-Gly-Asp- |
| ligand accessibility) | Val-Phe-Thr-Met-Pro- |
| Vitronectin Sequence | NH2 (MAA-PEO₄-SEQ |
| | ID NO: 28-NH₂) |
| Methacrylate Cyclic | Cyclo(Arg-Gly-Asp- |
| Peptide Constrained | Tyr-Lys)-Methacryl |
| Methacrylate Peptide | (SEQ ID NO: 34-MAA) |
| for increased stability | |

In embodiments of the present invention, a combination of monomers is provided on a substrate, including a functionalized polypeptide, a polypeptide containing a photo-polymerizable moiety, and the combination of monomers and functionalized polypeptide is polymerized in situ. Embodiments of the invention provide a process for coating in-situ cell adhesive photo-active moieties combined with photo-active hydrophilic monomers and cross-linkers to produce a peptide functionalized synthetic layer that can stimulate the growth and proliferation of difficult to culture cells such as human Embryonic Stem Cells (hESCs) in an undifferentiated state. In embodiments, these coatings contain peptides which are cell adhesive. In embodiments, the cell adhesive peptides have an RGD sequence. However, the peptides are not exclusive to peptides having the RGD peptide sequence. In embodiments methacrylate functionalized peptides are displayed on the surface interface and/or embedded in the matrix and are formulated in different molar concentrations while at the same time combining with different hydrophilic monomers to elicit different cell response. The photo-active or photo-polymerizable functionalized groups can include vitronectin and Laminin peptide sequences; they can be linear or cyclic and they can also contain at least two photo-active groups on the peptide that serve as cross-linkers or mimic a more constrained cyclic peptide structure when cross-linked.

Examples of embodiments of the mixtures of monomers and functionalized peptides used to form cell culture surfaces of the present invention are presented in Tables 3-8.

Table 3 shows dilution of BSP-methacrylate (MAA-SEQ ID NO:19) combined with HEMA as a hydrophilic monomer and TEGDMA as a hydrophilic cross-linker.

TABLE 3

| | HEMA-100- | | HEMA-75- | | HEMA-50- | | HEMA-25- | | HEMA-10- | |
|---|---|---|---|---|---|---|---|---|---|---|
| Formulation | BSP-MAA | | BSP-MAA | | BSP-MAA | | BSP-MAA | | BSP-MAA | |
| Hydroxyethyl Methacrylate (HEMA) | 400 | μL | 400 | μL | 400 | μL | 400 | μL | 400 | μL |
| Ac-Lys(MAA)-Gly-Gly-Asn-Gly-Glu-Pro- | 8.74 | mg | 6.55 | mg | 4.37 | mg | 2.19 | mg | 0.87 | mg |
| Arg-Gly-Asp-Thr-Tyr-Arg-Ala-Tyr-NH2 | | | | | | | | | | |
| (BSP-Methacrylate) | | | | | | | | | | |
| Formulation ID | PA1 | | PA2 | | PA3 | | PA4 | | PA5 | |
| Tetra(ethyleneglycol) dimethacrylate | 40 | μL | 40 | μL | 40 | μL | 40 | μL | 40 | μL |
| (TEGDA) | | | | | | | | | | |
| Darocur 1173 (10% in EtOH) | 15 | μL | 15 | μL | 15 | μL | 15 | μL | 15 | μL |
| Irgacure I-819 (1% in EtOH) | 50 | μL | 50 | μL | 50 | μL | 50 | μL | 50 | μL |
| Ethanol | 9.5 | mL | 9.5 | mL | 9.5 | mL | 9.5 | mL | 9.5 | mL |

Table 4 shows dilutions of VN-methacrylate (MAA-SEQ ID NO: 27) combined with HEMA as a hydrophilic monomer and TEGDMA as hydrophilic cross-linker.

TABLE 4

| | HEMA-100- | | HEMA-75- | | HEMA-50- | | HEMA-25- | | HEMA-10- | |
|---|---|---|---|---|---|---|---|---|---|---|
| Formulation | VN-MAA | | VN-MAA | | VN-MAA | | VN-MAA | | VN-MAA | |
| Hydroxyethyl Methacrylate (HEMA) | 400 | μL | 400 | μL | 400 | μL | 400 | μL | 400 | μL |
| Ac-Lys(MAA)-Gly-Gly-Pro-Gln-Val-Thr- | 8.74 | mg | 6.65 | mg | 4.37 | mg | 2.19 | mg | 0.87 | mg |
| Arg-Gly-Asp-Val-Phe-Thr-Met-Pro-NH2 | | | | | | | | | | |
| (VN-Methacrylate) | | | | | | | | | | |
| Formulation ID | PB1 | | 2PB2 | | PB3 | | PB4 | | PB5 | |
| Tetra(ethyleneglycol) dimethacrylate | 40 | μL | 40 | μL | 40 | μL | 40 | μL | 40 | μL |
| (TEGDMA) | | | | | | | | | | |
| Darocur 1173 (10% in EtOH) | 15 | μL | 15 | μL | 15 | μL | 15 | μL | 15 | μL |
| Irgacure I-819 (1% in EtOH) | 50 | μL | 50 | μL | 50 | μL | 50 | μL | 50 | μL |
| Ethanol | 9.5 | mL | 9.5 | mL | 9.5 | mL | 9.5 | mL | 9.5 | mL |

Table 5 shows formulations of a library showing dilution of VN-methacrylate with PEO spacer linker (MAA-PEG₄-SEQ ID NO: 27) combined with HEMA as a hydrophilic monomer and TEGDMA as a hydrophilic cross-linker.

TABLE 5

| | 100-HEMA- | | 75-HEMA- | | 50-HEMA- | | 25-HEMA- | | 10-HEMA- | |
|---|---|---|---|---|---|---|---|---|---|---|
| Formulation | MAA-PEG4-VN | | MAA-PEG4-VN | | MAA-PEG4-VN | | MAA-PEG4-VN | | MAA-PEG4-VN | |
| Hydroxyethyl Methacrylate (HEMA) | 400 | μL | 400 | μL | 400 | μL | 400 | μL | 400 | μL |
| MAA-(PEO)4-Lys-Gly-Gly-Pro-Gln-Val- | 4.37 | mg (RC1) | 3.28 | mg (RC2) | 2.19 | mg | 1.09 | mg | 0.437 | mg |
| Thr-Arg-Gly-Asp-Val-Phe-Thr-Met-Pro- | | | | | | | | | | |
| NH2 | | | | | | | | | | |
| Formulation ID | PC1 | | PC2 | | PC3 | | PC4 | | PC5 | |
| Tetra(ethyleneglycol) dimethacrylate | 40 | μL | 40 | μL | 40 | μL | 40 | μL | 40 | μL |
| TEGDMA | | | | | | | | | | |
| Darocur 1173 (10% in EtOH) | 15 | μL | 15 | μL | 15 | μL | 15 | μL | 15 | μL |
| Irgacure I-819 (1% in EtOH) | 50 | μL | 50 | μL | 50 | μL | 50 | μL | 50 | μL |
| Ethanol | 9.5 | mL | 9.5 | mL | 9.5 | mL | 9.5 | mL | 9.5 | mL |

Table 6 Shows formulation of a library showing dilution of VN-methacrylate with PEO spacer linker (MAA-PEG₄-SEQ ID NO: 27) combined with glycerol mono-methacrylate as a hydrophilic monomer and glycerol 1,3-diglycerolate dimethacrylate as a hydrophilic cross-linker.

TABLE 6

| | 100-GLY-MAA- | | 75-GLY-MAA- | | 50-GLY-MAA- | | 25-GLY-MAA- | | 10-GLY-MAA- | |
|---|---|---|---|---|---|---|---|---|---|---|
| Formulation | PEG4-VN | | PEG4-VN | | PEG4-VN | | PEG4-VN | | PEG4-VN | |
| Glycerol monomethacrylate | 400 | μL | 400 | μL | 400 | μL | 400 | μL | 400 | μL |
| MAA-PEG4-Lys-Gly-Gly-Pro-Glu-Val- | 4.37 | mg | 3.28 | mg | 2.19 | mg | 1.09 | mg | 0.437 | mg |
| Thr-Arg-Gly-Asp-Val-Phe-Thr-Met-Pro- | | | | | | | | | | |
| NH2 | | | | | | | | | | |
| Formulation ID | PD1 | | PD2 | | PD3 | | PD4 | | PD5 | |
| Glycerol 1,3-Diglycerolate | 40 | μL | 40 | μL | 40 | μL | 40 | μL | 40 | μL |
| Dimethacrylate | | | | | | | | | | |
| Darocur 1173 (10% in EtOH) | 15 | μL | 15 | μL | 15 | μL | 15 | μL | 15 | μL |
| Irgacure I-819 (1% in EtOH) | 50 | μL | 50 | μL | 50 | μL | 50 | μL | 50 | μL |
| Ethanol | 9.515 | mL | 9.515 | mL | 9.515 | mL | 9.515 | mL | 9.515 | mL |

Table 7 shows a library dilution of VN-monomethacrylate and VN dimethacrylate with PEO spacer linker (MAA-PEG₄-SEQ ID NO: 27) combined with HEMA as a hydrophilic monomer and TEGDMA as a hydrophilic cross-linker.

TABLE 7

| | HEMA-100- | | HEMA-75- | | HEMA-50- | | HEMA-25- | | HEMA-10- | |
|---|---|---|---|---|---|---|---|---|---|---|
| Formulation | VN-2MAA | | VN-2MAA | | VN-2MAA | | VN-2MAA | | VN-2MAA | |
| Hydroxyethyl Methacrylate (HEMA) | 400 | μL | 400 | μL | 400 | μL | 400 | μL | 400 | μL |
| Lys(MAA)-Gly-Gly-Pro-Gln-Val-Thr-Arg- | 8.74 | mg | 6.55 | mg | 4.37 | mg | 2.19 | mg | 0.87 | mg |
| Gly-Asp-Val-Phe-Thr-Met-Pro-NH2 (VN- | | | | | | | | | | |
| Methacrylate) | | | | | | | | | | |
| MAA-Lys-Gly-Gly-Asn-Gly-Glu-Pro-Arg- | 2.0 | mg | 1.5 | mg | 1.5 | mg | 1.0 | mg | 0.5 | mg |
| Gly-Asp-Thr-Tyr-Arg-Ala-Tyr-NH2-MAA | | | | | | | | | | |
| (BSP-Dimethacrylate) | | | | | | | | | | |
| Tetra(ethyleneglycol) dimethacrylate | 40 | μL | 40 | μL | 40 | μL | 40 | μL | 40 | μL |
| (TEGDMA) | | | | | | | | | | |
| Formulation ID | PE1 | | PE2 | | PE3 | | PE4 | | PE5 | |
| Darocur 1173 (10% in EtOH) | 15 | μL | 15 | μL | 15 | μL | 15 | μL | 15 | μL |
| Irgacure I-819 (1% in EtOH) | 50 | μL | 50 | μL | 50 | μL | 50 | μL | 50 | μL |
| Ethanol | 9.5 | mL | 9.5 | mL | 9.5 | mL | 9.5 | mL | 9.5 | mL |

Table 8 shows formulation of a library showing dilution of VN-methacrylate (MAA-SEQ ID NO: 27) combined with glyerol mono-methacrylate as a hydrophilic monomer and glycerol 1,3-diglycerolate dimethacryalte as a hydrophilic cross-linker.

TABLE 8

| | GLY-100- | | GLY-75- | | GLY-50- | | GLY-25- | | GLY-10- | |
|---|---|---|---|---|---|---|---|---|---|---|
| Formulation | VN-MAA | | VN-MAA | | VN-MAA | | VN-MAA | | VN-MAA | |
| Glycerol Monomethacrylate | 400 | μL | 400 | μL | 400 | μL | 400 | μL | 400 | μL |
| Ac-Lys(MAA)-Gly-Gly-Pro-Gln-Val-Thr-Arg- | 8.74 | mg | 6.55 | mg | 4.37 | mg | 2.19 | mg | 0.87 | mg |
| Gly-Asp-Val-Phe-Thr-Met-Pro-NH2 (VN- | | | | | | | | | | |
| Meth) | | | | | | | | | | |
| Formulation ID | PF1 | | PF2 | | PF3 | | PF4 | | PF5 | |
| Glycerol 1,3-Diglycerolate Dimethacrylate | 40 | μL | 40 | μL | 40 | μL | 40 | μL | 40 | μL |
| Darocur 1173 (10% in EtOH) | 15 | μL | 15 | μL | 15 | μL | 15 | μL | 15 | μL |
| Irgacure I-819 (1% in EtOH) | 50 | μL | 50 | μL | 50 | μL | 50 | μL | 50 | μL |
| Ethanol | 9.5 | mL | 9.5 | mL | 9.5 | mL | 9.5 | mL | 9.5 | mL |

The actual libraries tested showed a correlation between the amount of peptide present in the synthetic polymer matrix and cell response. It was observed that the increase in the overall quantity of peptide present in the matrix as demonstrated in surface HEMA-100-VN-2A, significantly increased cell adhesion and spreading.

In an additional embodiments, a cyclic functionalized polymer peptide surface was made using functionalized peptide (SEQ ID NO:34). The surface consisted of 1 mM (2.9 μg/cm²) of cyclo(Arg-Gly-Asp-D-Tyr-Lys(MAA)) (SEQ ID NO:34) combined with 2-hydroxyethylmethacrylate (a hydrophilic monomer) and a tetraethylene glycol dimethacrylate cross-linker present in 24.8 μg/cm² and 9.4 μg/cm²

Another hydrogel formulation that was prepared contained 80 μL of 2-hydroxyethyl Methacrylate and 30 μl, Tetra(ethylene glycol) Dimethacrylate. 30 μL of Darocur 1173 (10% in ethanol), 20 μL Irgacure 819 (1% in ethanol) and 9.92 ml of ethanol as a solvent. 1 mg of Cyclic RGD-VN-MAA was dissolved in 40 μL of 18 Mega OHM water in 10 μL increments. 960 μL of the acrylate hydrogel was added to the dissolved peptide for a total of 1 ml of a 1 mM peptide concentration solution.

In embodiments, methods of making peptide-containing polymeric cell culture surfaces are presented. In embodiments, the methods of making peptide-containing polymeric cell culture surfaces provide (1) mixing monomers including peptide-(meth)acrylate monomers; (2) providing the monomers to a cell culture substrate; (3) curing or polymerizing the monomers to form polymers; and, (4) washing. In embodiments, additional steps may include drying, providing a lid to the cell culture substrate, sterilizing, packaging and/or shipping the cell culture article having a peptide-containing polymeric cell culture surface. For example, in embodiments, a methacrylate containing peptide, in the presence of at least one (meth)acrylate monomer may be provided to a substrate.

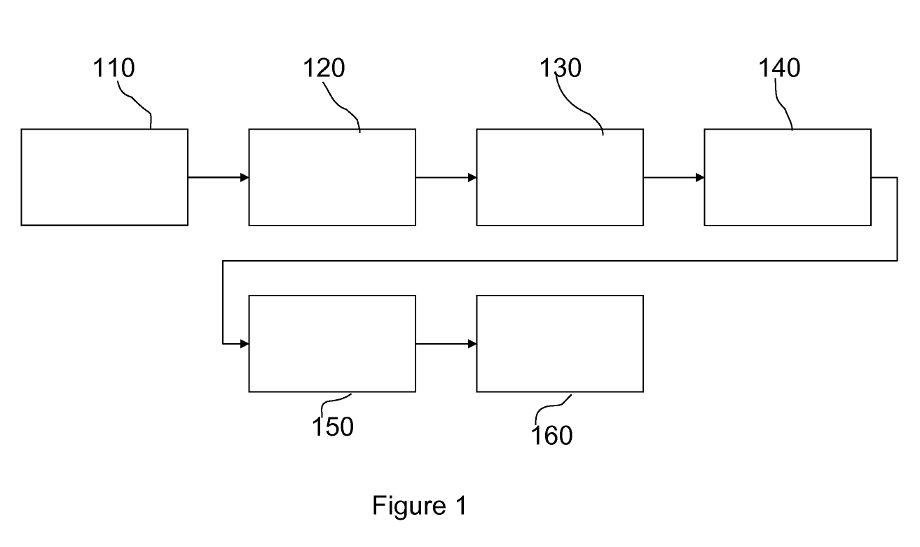

FIG. 1 is a flow chart showing an embodiment of a method of making cell culture surfaces. In embodiments, methods for providing cell binding peptides on the surface of a hydrophilic surface by photo-active chemical grafting are provided. These methods include steps of (110) mixing the functionalized peptide (ƒpeptide) with monomers; (120) apply mixture to a cell culture substrate; (130) polymerize the monomers and the functionalized peptide by, for example, exposure to UV/VIS energy to cure the monomers and functionalized peptide; (140) wash; (150) dry; (160) optional additional treatments such as applying a top to a topless flask (welding the top to the flask, for example), sterilization, labeling, packaging and shipping. In step (120) the mixture of functionalized peptide and monomer may be provided to the surface of a substrate by any means know in the art including liquid dispensing, spin coating, spray coating, or other methods. In step (130), the curing or polymerizing step may be accomplished by any means known in the art, and depending upon the nature of the polymerizing moiety, and may include the introduction of photoinitiators into the monomer mixture and the exposure of the surface to UV, visible or thermal energy. In step (140) washing may be accomplished by any means known in the art including liquid dispensing and incubating, with or without agitation, where the liquid may be water, a lower alcohol, a lower alcohol diluted in water, or other solvent. In step 150, drying may be accomplished by the application of a vacuum and/or heat. In step 160, a top may be provided to the cell culture substrate by any means known in the art including welding, heat sealing, pressure sealing, removably sealing, or any other method. Sterilization may occur by exposure to ethanol, for example, gamma irradiation, or other methods.

In embodiments, in step 110, addition to monomers, a composition forming the layer may include one or more additional compounds such as surfactants, wetting agents, photoinitiators, chain transfer agents, thermal initiators, catalysts and activators. Any suitable polymerization initiator may be employed. One of skill in the art will readily be able to select a suitable initiator, e.g. a radical initiator or a cationic initiator, suitable for use with the monomers. In various embodiments, UV light is used to generate free radical monomers to initiate chain polymerization. However, visible light initiators and low temperature initiators may be used instead of UV initiators to shield the peptide from exposure to a more harmful or damaging radiation source such as UV radiation.

Any suitable initiator may be used, including thermal initiators, photo-initiators or room temperature initiators. Examples of polymerization initiators include organic peroxides, azo compounds, quinones, nitroso compounds, acyl halides, hydrazones, mercapto compounds, pyrylium compounds, imidazoles, chlorotriazines, benzoin, benzoin alkyl ethers, diketones, phenones, or mixtures thereof. Potassium persulfate may be used as an initiator for room temperature polymerization. Examples of suitable commercially available, ultraviolet-activated and visible light-activated photoinitiators have tradenames such as IRGACURE 651, IRGACURE 184, IRGACURE 369, IRGACURE 819, DAROCUR 4265 and DAROCUR 1173 commercially available from Ciba Specialty Chemicals, Tarrytown, N.Y. and LUCIRIN TPO and LUCIRIN TPO-L commercially available from BASF (Charlotte, N.C.)

Additional initiators may include water soluble azo-initiators that can be used in thermal polymerization including, for example, (VA-044) 2,2′-Azobis[2-(2-imidazolin-2-yl)propane]dihydrochloride; (VA046B) 2,2′-Azobis[2-(2-imidazolin-2-yl)propane]disulfate dehydrate; (VA-50) 2,2′-Azobis(2-methylpropionamidine)dihydrochloride; (VA-057) 2,2′-Azobis[N-(2-carboxyethyl)-2-methylpropionamidine]hydrate; (VA-060) 2,2′-Azobis{2-[1-(2-hydroxyethyl)-2-imidazolin-2-yl]propane}dihydrochloride; (VA-061) 2,2′-Azobis[2-(2-imidazolin-2-yl)propane]; (VA-067) 2,2′-Azobis(1-imino-1-pyrrolidino-2-ethylpropane)dihydrochloride; (VA-080) 2,2′-Azobis{2-methyl-N-[1,1-bis(hydroxymethyl)-2-hydroxyethyl]propionamide or (VA-086) 2,2′-Azobis[2-methyl-N-(2-hydroxyethyl)propionamide]. Oil soluble azo-initiators such as (V-70) 2,2′-Azobis(4-methoxy-2,4-dimethyl valeronitrile); (V-65) 2,2′-Azobis(2,4-dimethyl valeronitrile); (V-601) Dimethyl 2,2′-azobis(2-methylpropionate); (V-59) 2,2′-Azobis(2-methylbutyronitrile; (V-40) 1,1′-Azobis(cyclohexane-1-carbonitrile); (VF-096) 2,2′-Azobis[N-(2-propenyl)-2-methylpropionamide]; (V-30) 1-[(1-cyano-1-methylethyl)azo]formamide; (VAm-110) 2,2′-Azobis(N-butyl-2-methylpropionamide) or (VAm-111) 2,2′-Azobis(N-cyclohexyl-2-methylpropionamide) may also be used in thermal polymerization. These initiators are available from for example, WAKO Chemicals, Richmond Va. In addition, macro-initiators, such as azo-initiators having a PEG backbone may be used in thermal polymerization.

A photosensitizer may also be included in a suitable initiator system. Representative photosensitizers have carbonyl groups or tertiary amino groups or mixtures thereof. Photosensitizers having a carbonyl groups include benzophenone, acetophenone, benzil, benzaldehyde, o-chlorobenzaldehyde, xanthone, thioxanthone, 9,10-anthraquinone, and other aromatic ketones. Photosensitizers having tertiary amines include methyldiethanolamine, ethyldiethanolamine, triethanolamine, phenylmethyl-ethanolamine, and dimethylaminoethylbenzoate. Commercially available photosensitizers include QUANTICURE ITX, QUANTICURE QTX, QUANTICURE PTX, QUANTICURE EPD from Biddle Sawyer Corp., Crawley, England.

In general, the amount of photosensitizer or photoinitiator system may vary from about 0.01 to 10% by weight.

Examples of cationic initiators include salts of onium cations, such as arylsulfonium salts, as well as organometallic salts such as ion arene systems.

In embodiments, the substrate may be any material suitable for culturing cells, including a ceramic substance, a glass, a plastic, a polymer or co-polymer, any combinations thereof, or a coating of one material on another. The substrate may be flat or shaped. Such substrates include glass materials such as soda-lime glass, pyrex glass, vycor glass, quartz glass; silicon; plastics or polymers, including dendritic polymers, such as poly(vinyl chloride), poly(vinyl alcohol), poly(methyl methacrylate), poly(vinyl acetate-co-maleic anhydride), poly(dimethylsiloxane) monomethacrylate, cyclic olefin polymers, fluorocarbon polymers, polystyrenes, polypropylene, polyethyleneimine; copolymers such as poly(vinyl acetate-co-maleic anhydride), poly(styrene-co-maleic anhydride), poly(ethylene-co-acrylic acid) or derivatives of these or the like. As used herein, “cyclic olefin copolymer” means a polymer formed from more than one monomer species, where at least one of the monomer species is a cyclic olefin monomer and at least one other monomer species is not a cyclic olefin monomer species. In many embodiments, cyclic olefin copolymers are formed from ethylene and norbonene monomers. Cyclic olefin copolymer resins are commercially available with trade name of TOPAS® from Boedeker Plastics, Inc., Japan and Zeonor from Zeon Chemicals, L.P. Lousiville, Ky. In embodiments, the substrate may be treated to enhance retention of the polymer matrix. For example, the substrate may be treated with chemical or plasma treatments which provide negative charge, positive charge, create a more hydrophilic surface, or create functional chemical groups that enhance the adhesion of the polymer matrix to the substrate. For example, such treatments may include hydrophobic or hydrophilic interactions, steric interactions, affinities or Vander Waal forces.

To form the peptide functionalized synthetic cell culture layer, the monomers and the functionalized peptide are polymerized. Whether polymerized in bulk phase (substantially solvent free) or solvent phase, the monomers are polymerized via an appropriate initiation mechanism. Many of such mechanisms are known in the art. For example, temperature may be increased to activate a thermal initiator; photoinitiators may be activated by exposure to appropriate wavelength of light, or the like. According to numerous embodiments, the monomer or monomer mixture is cured using UV light. The curing preferably occurs under inert gas protection, such as nitrogen protection, to prevent oxygen inhibition. Suitable UV light combined with gas protection may increase polymer conversion, insure coating integrity and reduce cytotoxicity.

In embodiments, the layer may be washed with solvent one or more times to remove impurities such as unreacted monomers or low molecular weight polymer species. In various embodiments, the layer is washed with ethanol or an ethanol-water solution, e.g. 70% ethanol, greater than 90% ethanol, greater than 95% ethanol or greater than about 99% ethanol. Washing with a 70% ethanol solvent may not only serve to remove impurities, which may be cytotoxic, but also can serve to sterilize the surface prior to incubation with cells.

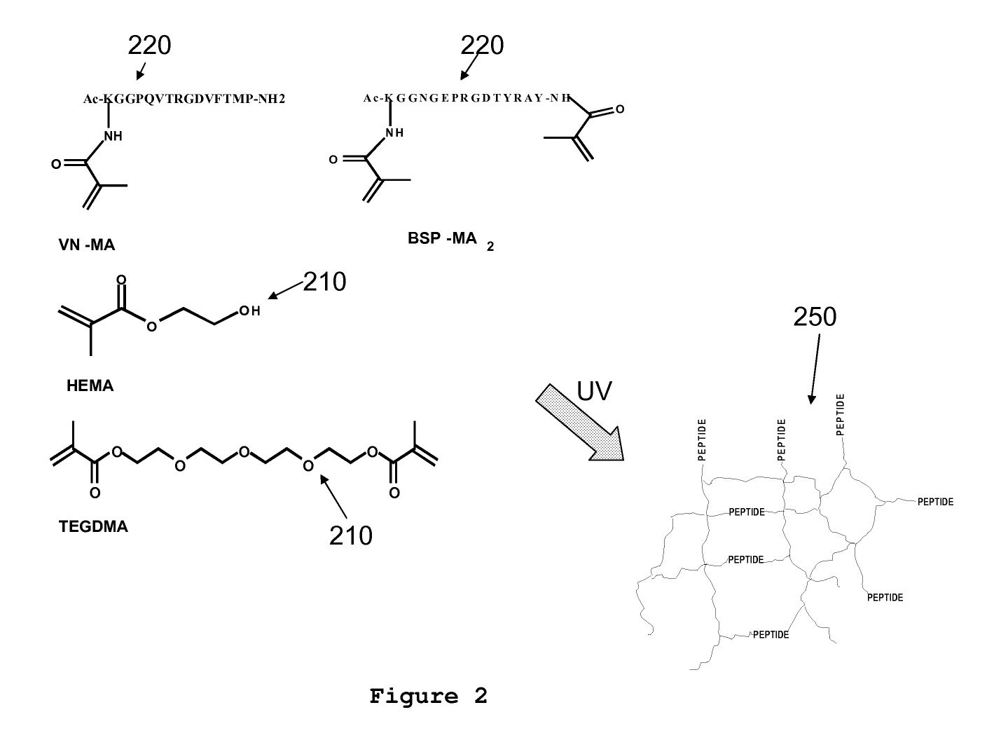

FIG. 2 is an illustration showing a method for making an embodiment of a cell culture surface. In FIG. 2, acrylate or methacrylate monomers 210 such as, for example, Tetraethylene glycol dimethacrylate (TEGDMA) and hydroxylethyl methacrylate (FFEMA) are mixed with a functionalized peptide 220 such as VN-Methacrylate or BSP-(methacrylate)₂ or a mixture of functionalized peptides 220, applied to a substrate and exposed to UV radiation to form a peptide conjugated polymer 250 surface suitable for cell culture applications. Referring to Formula 1, VN-Methacrylate is Aₘ-S_{y}-Xaaₙ-S_{y1}-Z-S_{y2}Aₘ₁ Xaa is Lys and is acetylated, n=1, “A” is methacrylic acid (MAA) and m=1, the spacer, S_{y}, is absent (y=0), the spacer S_{y1} is absent (y1=0), peptide Z is Seq ID NO: 28 and spacer Sy2 is absent (y2=0) and the Am1 is absent (m1=0). Referring to Formula 1, BSP-(Methacrylate)₂ is: Aₘ-S_{y}-Xaaₙ-S_{y1}-Z-S_{y2}-Aₘ₁, where “Aₘ” is methacrylic acid (MAA) (m=1), the spacer, S_{y}, is absent (y=0), Xaa is Lys and is acetylated (n=1), Spacer S_{y1} is absent y1=0), the peptide Z is SEQ ID NO: 19, spacer S_{y2} is absent (y2=0) and “Aₘ₂” is methacrylic acid or any carboxylic acid functionalized methacrylate. In this example, a second methacrylic acid moiety is present at the N terminal end of the peptide sequence via a c-linker.

FIG. 2 illustrates a reaction scheme showing VN-Methacrylate and/or BSP-(methacrylate)₂ combined with HEMA as a hydrophilic monomer and TEGDMA (triethylene glycol dimethacrylate) as a cross-linker forming a polymer matrix containing peptide in the polymer matrix upon exposure to UV energy (in the presence of a photopolymerizing agent such as 1-819 or D-1173).

Without being limited by theory, it may be that the cross-linking ability of the VN-dimethacrylate created a more stable constrained arrangement in the matrix that may have influenced a positive cell response. Overall, there was an increase in cell number of 2-3 times more for HEMA-100-VN-2A surface when compared with those other surfaces that had less peptide density.

In embodiments, a series of functionalized mono and di methacrylated peptides that are reacted by free radical photo-polymerization in-situ with, for example, hydroxylethyl methacrylate and tetraethylene glycol dimethacrylate to form a cross-linked polymer-peptide matrix are provided. Other monomers that are more hydrophilic are also presented in the formulation library. Furthermore, this invention allows for very low feed concentration of peptide into the matrix as well as allowing for over 95% utilization of the photo-active peptide. This represents a significant improvement over previous methods. Specifically, embodiments of methods of the present invention utilizes, for example 0.75 mL-1.5 mL of monomer-peptide solution which is equivalent to 0.63 mg to 2.4 mg when spun coated in a T75 flask while previous methods required 16.8 mg of peptide and only utilized less than 1% in the same format as mentioned previously. This invention also allows extension to other coating techniques beyond spin coating such as ultrasonic spray coating, dip coating and solution casting with maximum material utilization. Another critical attribute is the ability to use this method with other substrates including thermoplastic resin without the incidence of chemical interaction with the surface of the plastic. It also removes DMF solvent from the process, therefore facilitating the use of cheaper thermoplastic resin as the base substrate, and reducing the burden of disposing of DMF after use.

Another benefit of embodiments of methods of the present invention is a significant reduction in process steps which have a large impact on the overall manufacturing cost. This method provides a novel, facile synthetic pathway to producing chemically linked peptide surfaces for cell culture.

In embodiments, the hydrophilic monomers are, for example, glycerol monomethacrylate (GLY-METH), Glycerol 1,3-diglycerolate dimethacrylate (DGDMA), or hydroxyethyl methacrylate (HEMA), or tetraethylene glycol dimethacrylate (TEGDM). Acrylate and methacrylate monomers may be synthesized as known in the art or obtained from a commercial vendor, such as Polysciences, Inc., Warrigton, Pa. Sigma Aldrich, Inc., St. Louis, Mo. and Sartomer, Inc., Exton, Pa. Polypeptides may be synthesized as known in the art (or alternatively produced through molecular biological techniques) or obtained from a commercial vendor, such as American Peptide Corporation, Sunnyvale, Calif., Gen GenScript Corporation, Piscataway, N.J. and Genway Biotechnology, San Diego, Calif. Spacers may be synthesized as known in the art or obtained from a commercial vendor, such as discrete polyethylene glycol (dPEG) spacers available from Quanta BioDesign, Ltd., Powell, Ohio. Embodiments of the cell culture surface of the present invention are shown in Tables 3-8.

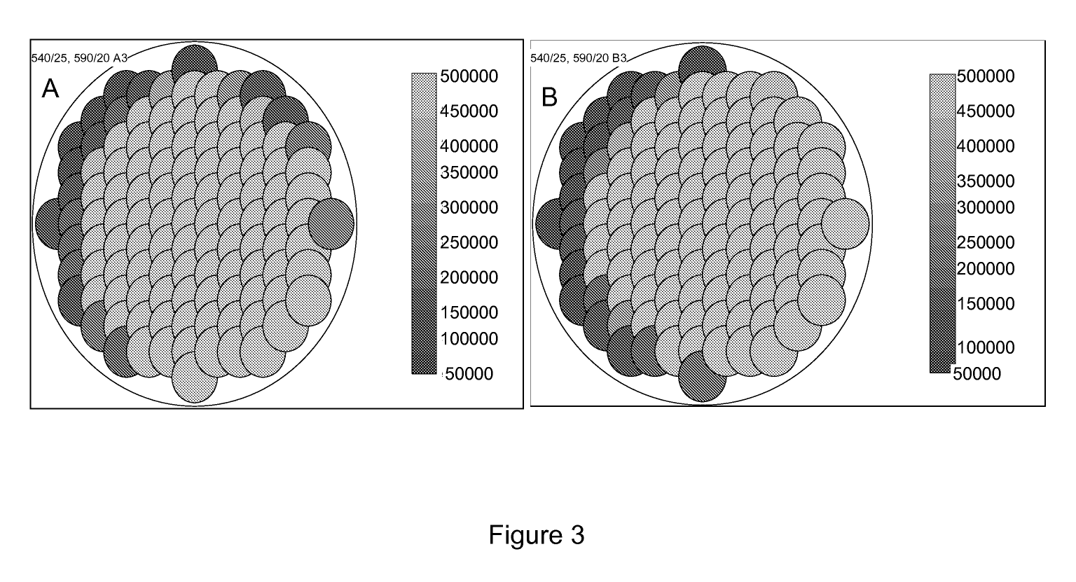

FIG. 3 shows the fluorescence intensity of fluorescently labeled peptide polymer distributed over a cell culture surface. In this figure Rhodamine labeled Ac-Lys-(MAA)-Gly-Gly-Val-Thr-Arg-Gly-Asp-Val-Phe-Thr-Met-Pro-NH2-TAMRA (Ac-K-MAA-SEQ ID NO:35-TAMRA) spiked in 0.2% in unlabeled methacrylate peptide sequence formulated with HEMA and TEGDMA. TAMRA is tetramethyl rhodamine dye which is used to label the peptide. FIG. 3 illustrates that the coatings are relatively uniform except for slight non-uniformity on the edges.

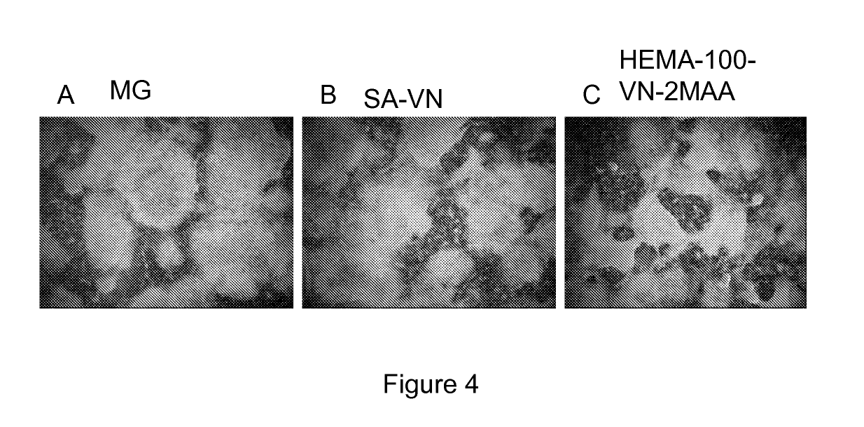

FIG. 4 shows photomicrographs of H7 human embryonic stem cells cultured for four days in six well plates on an embodiment of the present invention (FIG. 4C) compared to H7 human embryonic stem cells cultured on control surfaces Matrigel™ available form BD, Franklin Lakes, N.J., (FIG. 4A) and Synthemax™ available from Corning Incorporated, Corning, N.Y. (FIG. 4B), a vitronectin peptide conjugated swellable acrylate surface made according to the methods described in copending application Ser. Nos. 12/362,974 and 12/362,782, both incorporated by reference herein in their entirety. As shown in FIG. 4, the morphology of these cells grown on an embodiment of the present invention is similar to the morphology of these cells on control surfaces, although cells grown on an embodiment of the present invention have a more cystic morphology when compared to controls.

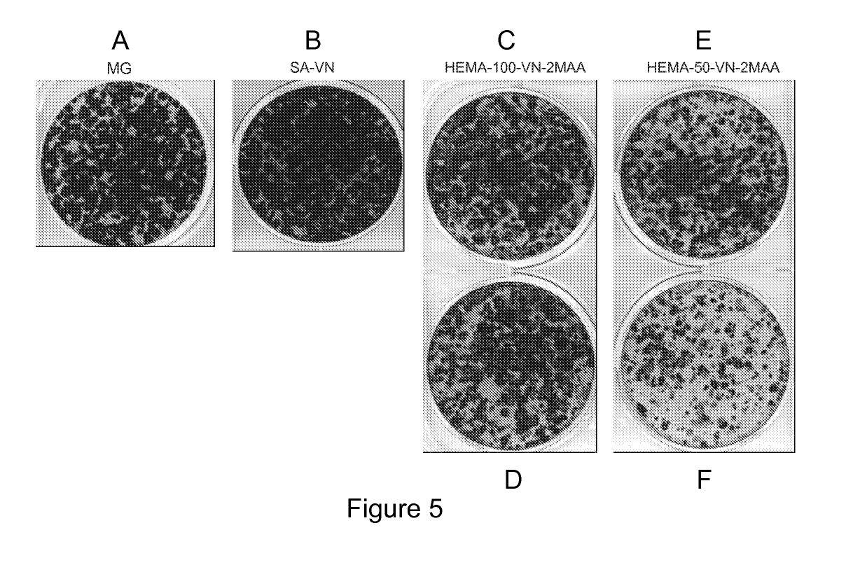

FIG. 5 shows photomicrographs of crystal violet-stained H7 human embryonic stem cells cultured on functionalized peptide-polymer coated surfaces in embodiments of the present invention compared to control surfaces. Crystal violet-stained H7 hESCs on: (FIG. 5A) Matrigel™, (FIG. 5B) Synthemax™, (FIGS. 5C and D) HEMA-100-VN-2MAA, and (FIGS. 5E and F) HEMA-50-VN-2MAA as described in Table 7 above. FIGS. 5D and F are duplicate experiments shown for HEMA-100-VN-2MAA and HEMA-50-VN-2MAA. FIG. 5 illustrates that H7 hESC cells grown on embodiments of the present invention are comparable to those grown on positive control surfaces (Matrigel™ and Synthemax™).

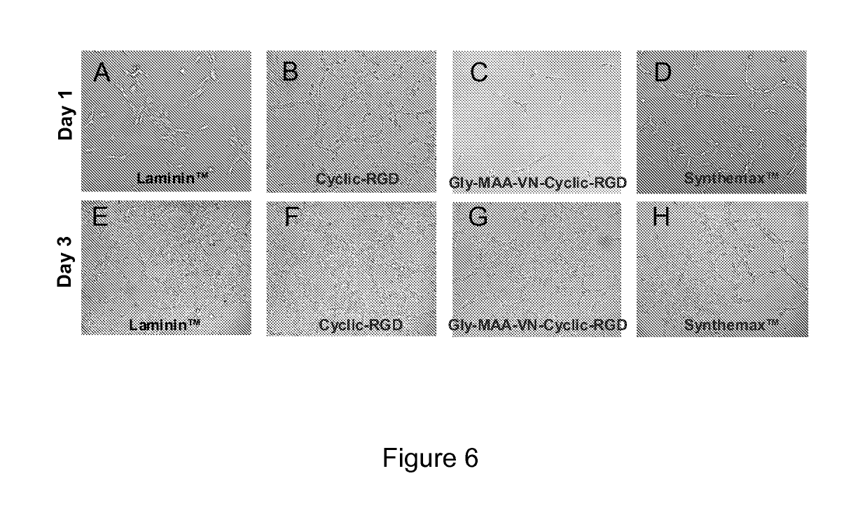

FIG. 6A-H show photomicrographs of neural progenitor cells, after one day in culture (FIGS. 6 A-D) and after three days in culture (FIG. 6E-H) growing on Laminin™ surface from Corning, Incorporated (FIGS. 6A and E), on a cyclic functionalized RGD peptide surface made with HEMA and TEGDMA (FIGS. 6B and F), on a cyclic functionalized RGD peptide (cyclic SEQ ID NO: 34) surface made with glycerol methacrylate and 1,3-diglycerolate dimethacrylate (FIGS. 6C and G) and on a Synthemax™ surface from Corning Incorporated (FIGS. 6D and H).

In embodiments, the cell culture surface may be formed on any surface suitable for cell culture. Examples of articles suitable for cell culture include single and multi-well plates, such as 6, 12, 96, 384, and 1536 well plates, jars, petri dishes, flasks, beakers, plates, roller bottles, slides, such as chambered and multichambered culture slides, tubes, cover slips, bags, membranes, hollow fibers, beads and microcarriers, cups, spinner bottles, perfusion chambers, bioreactors, CellSTACK® (Corning, Incorporated) and fermenters.

Cells may be used for any suitable purpose, including (i) obtaining sufficient amounts of undifferentiated stem cells cultured on a synthetic surface in a chemically defined medium for use in investigational studies or for developing therapeutic uses, (ii) for investigational studies of the cells in culture, (iii) for developing therapeutic uses, (iv) for therapeutic purposes, (v) for studying gene expression, e.g. by creating cDNA libraries, and (vi) for studying drug and toxicity screening.

Cell culture articles prepared according to embodiments of the methods of the present invention can be effectively presented to facilitate growth and proliferation of any relevant cell type, including, primary cells, cell lines, tissues and, for example, stem cells, adult stem cells, Embryonic Stem Cells (ESCs), human Embryonic Stem Cells (hESCs) or Inducible Pluripotent cells (IPCs). In embodiments, these cells in culture may be used in therapeutic applications. Because human embryonic stem cells (hESC) have the ability to grown continually in culture in an undifferentiated state, the hESC for use in this invention may be obtained from an established cell line. Examples of human embryonic stem cell lines that have been established include, but are not limited to, H1, H7, H9, H13 or H14 (available from WiCell established by the University of Wisconsin) (Thompson (1998) Science 282:1145); hESBGN-01, hESBGN-02, hESBGN-03 (BresaGen, Inc., Athens, Ga.); HES-1, HES-2, HES-3, HES-4, HES-5, HES-6 (from ES Cell International, Inc., Singapore); HSF-1, HSF-6 (from University of California at San Francisco); I 3, I 3.2, I 3.3, I 4, I 6, I 6.2, J 3, J 3.2 (derived at the Technion-Israel Institute of Technology, Haifa, Israel); UCSF-1 and UCSF-2 (Genbacev et al., Fertil. Steril. 83(5):1517-29, 2005); lines HUES 1-17 (Cowan et al., NEJM 350(13):1353-56, 2004); and line ACT-14 (Klimanskaya et al., Lancet, 365(9471):1636-41, 2005). Embryonic stem cells used in the invention may also be obtained directly from primary embryonic tissue. Typically this is done using frozen in vitro fertilized eggs at the blastocyst stage, which would otherwise be discarded.

Other suitable stem cells include induced primate pluripotent (iPS) stem cells OPCs according to the invention may also be differentiated from induced primate pluripotent stem (iPS) cells. iPS cells refer to cells, obtained from a juvenile or adult mammal, such as a human, that are genetically modified, e.g., by transfection with one or more appropriate vectors, such that they are reprogrammed to attain the phenotype of a pluripotent stem cell such as an hESC. Phenotypic traits attained by these reprogrammed cells include morphology resembling stem cells isolated from a blastocyst as well as surface antigen expression, gene expression and telomerase activity resembling blastocyst derived embryonic stem cells. The iPS cells typically have the ability to differentiate into at least one cell type from each of the primary germ layers: ectoderm, endoderm and mesoderm and thus are suitable for differentiation into a variety of cell types. The iPS cells, like hESC, also faun teratomas when injected into immuno-deficient mice, e.g., SCID mice. (Takahashi et al., (2007) Cell 131(5):861; Yu et al., (2007) Science 318:5858).

Embodiments of the present invention provide for efficient techniques for creating functionalized peptide-polymer coated surfaces in a cost effective manner and facile manufacturing processes leading to overall significant reduction in manufacturing costs. In embodiments, the surfaces are useful surfaces for culturing cells, including human embryonic stem cells in the pluripotent (having more than one potential outcome) state using chemically defined media. The use of chemically defined media, in combination with synthetic surfaces in embodiments of the present invention is important because the use of serum introduces undefined factors into cell culture which may be detrimental to cultured cells intended for therapeutic uses.

In an aspect (1), a functionalized peptide monomer wherein the functionalized peptide monomer is described by the formula: Aₘ-S_{y}-Xaaₙ-S_{y1}-Z-S_{y2}Aₘ₁ where A is a polymerization moiety, m is an integer from 1 to 6, m1 is an integer from 1 to 6, Xaaₙ is independently any amino acid, n is an integer from 0 to 6, S is a spacer, y is an integer from 0 to 30, y1 is an integer from 0 to 30, y2 is an integer from 0 to 30, and Z is a cell adhesive peptide is provided. In an aspect (2), the functionalized peptide monomer of aspect 1 is provided wherein the cell adhesive peptide (Z) comprises the sequence: KGGGQKCIVQTTSWSQCSKS (SEQ ID NO:1); GGGQKCIVQTTSWSQCSKS(SEQ ID NO:2); KYGLALERKDHSG (SEQ ID NO:3); YGLALERKDHSG (SEQ ID NO:4); KGGSINNNRWHSIYITRFGNMGS (SEQ ID NO:5); GGSINNNRWHSIYITRFGNMGS (SEQ ID NO:6); KGGTWYKITAFQRNRK (SEQ ID NO:7); GGTWYKIAFQRNRK (SEQ ID NO:8); KGGTSIKIRGTYSER (SEQ ID NO:9); GGTSIKIRGTYSER (SEQ ID NO:10); KYGTDIRVTLNRLNTF (SEQ ID NO:11); YGTDIRVTLNRLNTF (SEQ ID NO:12); KYGSETTVKYIFRLHE (SEQ ID NO:13); YGSETTVKYIFRLHE (SEQ ID NO:14); KYGKAFDITYVRLKF (SEQ ID NO:15); YGKAFDITYVRLKF (SEQ ID NO:16); KYGAASIKVAVSADR (SEQ ID NO:17); YGAASIKVAVSADR(SEQ ID NO:18); KGGNGEPRGDTYRAY(SEQ ID NO:19); GGNGEPRGDTYRAY (SEQ ID NO:20) CGGNGEPRGDTYRAY (SEQ ID NO:21); GGNGEPRGDTRAY (SEQ ID NO:22); KYGRKRLQVQLSIRT (SEQ ID NO:23); YGRKRLQVQLSIRT (SEQ ID NO:24); KGGRNIAEIIKDI (SEQ ID NO:25); GGRNIAEIIKDI (SEQ ID NO:26); KGGPQVTRGDVFTMP (SEQ ID NO:27); GGPQVTRGDVFTMP (SEQ ID NO:28); GGPQVTRGDVFTMPK (SEQ ID NO:29); GRGDSPK (SEQ ID NO:30); KGGAVTGRGDSPASS(SEQ ID NO:31); GGAVTGRGDSPASS (SEQ ID NO:32); Yaa₁PQVTRGNVFTMP (SEQ ID NO:32); RGDYK (SEQ ID NO:34) where SEQ ID NO: 34 may be straight or cyclic), or combinations. In an aspect (3), the functionalized peptide monomer of aspect 1 or 2 is provided, wherein the cell adhesive peptide comprises KGGPQVTRGDVFTMP (SEQ ID NO:27) or GGPQVTRGDVFTMP (SEQ ID NO:28). In an aspect (4), the functionalized peptide of any one of aspects 1-3 is provided wherein the polymerization moiety A comprises an acrylate or a methacrylate moiety. In an aspect (5), the functionalized peptide of any one of aspects 1-4 is provided wherein the polymerization moiety is methacrylic acid. In an aspect (6) the functionalized peptide of any one of aspects 1-5 is provided wherein the spacer is a polyethylene oxide with 20 or fewer repeating units. In an aspect (7), the functionalized peptide of any one of aspects 1-6 is provided wherein the spacer is PEG₄.

In an additional aspect (8), a polymer formed from a mixture comprising the functionalized peptide of any one of aspects 1-7 and at least one photopolymerizable monomer is provided. In an aspect (9), the polymer of aspect 8 is provided wherein the at least one photopolymerizable monomer is HEMA, TEGDMA, glycerol monomethacrylate or glycerol 1,3-diglycerolate dimethacrylate. In an aspect (10), the polymer of aspect 8 or 9 is provided wherein the polymer is formed from a mixture comprising a functionalized peptide, HEMA and TEGDA. In an aspect (11), the polymer of any one of aspects 8-10 is provided wherein the polymer is formed from a mixture comprising a functionalized peptide, Glycerol monomethacrylate and glycerol 1,3-diglycerolate dimethacrylate. In an aspect (12), the polymer of any one of aspects 8-11 is provided wherein the polymer is fowled in situ.

In an additional aspect (13), a method of making a cell culture surface is provided, comprising the steps of: providing a mixture of functionalized peptide monomer and at least one (meth)acrylate monomer; applying the mixture to a cell culture substrate; and polymerizing the monomers in situ to form a peptide-polymer. In an aspect (14), the method of aspect 13 is provided wherein the (meth)acrylate monomer comprises HEMA, TEGDA, Glycerol monomethacrylate or glycerol 1,3-diglycerolate dimethacrylate or combinations. In an aspect (15), the method of claim 13 or 14 is provided wherein the methacrylate monomer comprises a combination of HEMA and TEGDA. In an aspect (16), the method of any one of aspects 13-15 is provided wherein the functionalized peptide monomer is described by the formula: Aₘ-S_{y}-Xaaₙ-S_{y1}-Z-S_{y2}-Aₘ₁ where A is a polymerization moiety, m is an integer from 1 to 6, m1 is an integer from 1 to 6, Xaaₙ is independently any amino acid, n is an integer from 0 to 6, S is a spacer, y is an integer from 0 to 30, y1 is an integer from 0 to 30, y2 is an integer from 0 to 30, and Z is a cell adhesive peptide. In an aspect (17), the method of any one of aspects 13-16 is provided wherein Xaa is Lys and n=1. In an aspect (18), the method of any one of aspect 13-17 is provided wherein A is an acrylate or methacrylate. In an aspect (19), the method of any one of aspects 13-18 is provided wherein S is PEO and m=4. In an aspect (20), the method of any one of aspects 13-19 is provided wherein Z is a cell adhesive peptide selected from the group consisting of: KGGGQKCIVQTTSWSQCSKS (SEQ ID NO:1); GGGQKCIVQTTSWSQCSKS(SEQ ID NO:2); KYGLALERKDHSG (SEQ ID NO:3); YGLALERKDHSG (SEQ ID NO:4); KGGSINNNRWHSIYITRFGNMGS (SEQ ID NO:5); GGSINNNRWHSIYITRFGNMGS (SEQ ID NO:6); KGGTWYKIAFQRNRK (SEQ ID NO:7); GGTWYKIAFQRNRK (SEQ ID NO:8); KGGTSIKIRGTYSER (SEQ ID NO:9); GGTSIKIRGTYSER (SEQ ID NO:10); KYGTDIRVTLNRLNTF (SEQ ID NO:11); YGTDIRVTLNRLNTF (SEQ ID NO:12); KYGSETTVKYIFRLHE (SEQ ID NO:13); YGSETTVKYIFRLHE (SEQ ID NO:14); KYGKAFDITYVRLKF (SEQ ID NO:15); YGKAFDITYVRLKF (SEQ ID NO:16); KYGAASIKVAVSADR (SEQ ID NO:17); YGAASIKVAVSADR(SEQ ID NO:18); KGGNGEPRGDTYRAY(SEQ ID NO:19); GGNGEPRGDTYRAY (SEQ ID NO:20) CGGNGEPRGDTYRAY (SEQ ID NO:21); GGNGEPRGDTRAY (SEQ ID NO:22); KYGRKRLQVQLSIRT (SEQ ID NO:23); YGRKRLQVQLSIRT (SEQ ID NO:24); KGGRNIAEIIKDI (SEQ ID NO:25); GGRNIAEIIKDI (SEQ ID NO:26); KGGPQVTRGDVFTMP (SEQ ID NO:27); GGPQVTRGDVFTMP (SEQ ID NO:28); GGPQVTRGDVFTMPK (SEQ ID NO:29); GRGDSPK (SEQ ID NO:30); KGGAVTGRGDSPASS(SEQ ID NO:31); GGAVTGRGDSPASS (SEQ ID NO:32); Yaa₁PQVTRGNVFTMP (SEQ ID NO:32); RGDYK (SEQ ID NO:34), where the peptide sequences may be linear or cyclic, or combinations. In an aspect (21), a cell culture surface made by the method of any one of aspects 13-20 is provided.

In another aspect (22), a functionalized cyclic peptide monomer wherein the functionalized peptide monomer is described by the formula: Z_{c}-S_{y}-Xaaₙ-Aₘ wherein Zc is a cell adhesive cyclic peptide, S is a spacer, y is an integer from 0 to 30, Xaaₙ is independently any amino acid, n is an integer from 0 to 6, A is a polymerization moiety and m is an integer from 1 to 6 is provided. In an aspect (23) the functionalized cyclic peptide monomer of aspect 22 wherein the polymerization moiety A comprises an acrylate or a methacrylate moiety. In an aspect (24), the functionalized cyclic peptide of aspect 22 or 23 is provided wherein the polymerization moiety is methacrylic acid. In an aspect (25), the functionalized cyclic peptide of any one of aspect 22-24 is provided wherein the spacer is a polyethylene oxide with 20 or fewer repeating units. In an aspect (26), the functionalized cyclic peptide of any one of aspect 22-25 is provided wherein the spacer is PEG₄.

In another aspect (27) a polymer formed from a mixture comprising the functionalized cyclic peptide of any one of aspects 22-26 and at least one photopolymerizable monomer is provided. In an aspect (28), the polymer of aspect 27 is provided wherein the at least one photopolymerizable monomer is HEMA, TEGDMA, glycerol monomethacrylate or glycerol 1,3-diglycerolate dimethacrylate. In an aspect (29), the polymer of aspect 27 or 28 is provided wherein the polymer is formed from a mixture comprising a functionalized peptide, HEMA and TEGDA. In an aspect (30), the polymer of any one of aspects 27-29 is provided, wherein the polymer is formed from a mixture comprising a functionalized peptide, Glycerol monomethacrylate and glycerol 1,3-diglycerolate dimethacrylate.

In an aspect, a method of culturing human embryonic stem cells on a cell culture surface formed from the polymer according to any one of aspects 27-30 is provided comprising providing human embryonic stem cells in a cell culture medium to the polymer, and incubating the cells on the polymer. In another aspect, a method of culturing neuronal progenitor stem cells on a cell culture surface formed from the polymer according to any one of aspects 27-30 is provided comprising providing neuronal progenitor stem cells in a cell culture medium to the polymer, and incubating the cells on the polymer.

In the following, non-limiting examples are presented, which describe various embodiments of the articles and methods discussed above.

### Examples

Abbreviations: (BSP-Methacrylate), Ac-Lys(MAA)-Gly-Gly-Asn-Gly-Glu-Pro-Arg-Gly-Asp-Thr-Tyr-Arg-Ala-Tyr-NH₂ or Ac-K-MAA-SEQ ID NO:19-NH₂; (VN-Methacrylate), -Ac-Lys(MAA)-Gly-Gly-Pro-Gln-Val-Thr-Arg-Gly-Asp-Val-Phe-Thr-Met-Pro-NH₂ or Ac-K-MAA-SEQ ID NO:27-NH₂, (MAA-PEG₄-VN), MAA-PEG₄-Lys-Gly-Gly-Pro-Gln-Val-Thr-Arg-Gly-Asp-Val-Phe-Thr-Met-Pro-NH₂ or MAA-PEG₄-SEQ ID NO:27, (BSP-DIMAA), MAA-Lys-Gly-Gly-Asn-Gly-Glu-Pro-Arg-Gly-Asp-Thr-Tyr-Arg-Ala-Tyr-NH₂-MAA or MAA-SEQ ID NO:19-MAA, (GDGMDMA)—Glycerol 1,3-Diglycerolate Dimethacrylate, (GMMA)-Glycerol monomethacrylate, TEGDMA—Tetraethyleneglycol dimethacrylate, HEMA—2-hydroxyethyl methacrylate, EtOH—Ethanol, I-819, Darocur 1173.

Materials: Photoinitiators Irgacure-819 (Phosphine oxide, phenyl bis(2,4,6-trimethyl benzoyl) and Darocur 1173 (2-hydroxy-2-methyl-1-phenyl-1-propanone) used in the free radical polymerization of the acrylate hydrogel formulations were obtained from Ciba Specialty Chemicals (Newport Del.) and used without any further purification. Hydrophilic crosslinkers, tetraethylene glycol dimethacrylate (86680), (454982) and glycerol 1,3-diglycerol diacrylate (475807) were all purchased from Sigma-Aldrich in the purity as described in product specification sheet. Hydrophilic monomers 2-hydroxyethylmethacrylate, +99% (477028) was purchased from Sigma-Aldrich while the other hydrophilic monomer used in the formulations, glycerol monomethacrylate isomers (04180) was purchased from Polysciences Incorporated without further purification. Ac-Lys(MAA)-Gly-Gly-Asn-Gly-Glu-Pro-Arg-Gly-Asp-Thr-Tyr-Arg-Ala-Tyr-NH₂ (SEQ ID NO:19), Ac-Lys(MAA)-Gly-Gly-Pro-Gln-Val-Thr-Arg-Gly-Asp-Val-Phe-Thr-Met-Pro-NH₂ (SEQ ID NO:27), or (MAA-PEG₄-VN), MAA-PEG₄-Lys-Gly-Gly-Pro-Gln-Val-Thr-Arg-Gly-Asp-Val-Phe-Thr-Met-Pro-NH₂ (SEQ ID NO:27), Ac-Lys(MAA)-Gly-Gly-Asn-Gly-Glu-Pro-Arg-Gly-Asp-Thr-Tyr-Arg-Ala-Tyr-NH₂ (SEQ ID NO:19), MAA-Lys(MAA)-Gly-Gly-Pro-Gln-Val-Thr-Arg-Gly-Asp-Val-Phe-Thr-Met-Pro-NH₂ (SEQ ID NO:27). C[Arg-Gly-Asp-DTyr-Lys]-[MAA] (SEQ ID NO:34) or c(RGDyK) (SEQ ID NO: 34) were all purchased from American Peptide Corporation, Sunnyvale, Calif. and were synthesized by the following process:

### General Process for the Synthesis of Functionalized Peptides:

Preparation of Ac-Lys(MAA)-Gly-Gly-Pro-Gln-Val-Thr-Arg-Gly-Asp-Val-Phe-Thr-Met-Pro-NH₂ (SEQ ID NO:27): The peptide was synthesized on 15 mmol Fmoc-Rink Amide resin via Fmoc chemistry. Protecting groups used for amino acids are: t-Butyl group for and Asp and Thr, Trt group for Gln, Pbf for Arg, Ivdde for Lys. Fmoc protected amino acids were purchased from EMD Biosciences. Reagents for coupling and cleavage were purchased from Aldrich. Solvents were purchased from Fisher Scientific. The peptide chain was assembled on resin by repetitive removal of the Fmoc protecting group and coupling of protected amino acid. DIC and HOBt were used as coupling reagents and NMM was used as base. 20% piperidine in DMF was used as de-Fmoc-reagent. Methacrylic acid (MAA) was coupled on the side chain of Lysine after Ivdde was removed by 2% Hydrazine in DMF. After the last coupling, resin was treated with TFA/TIS/H2O (95:3:2, v/v/v) for cleavage and removal of the side chain protecting groups. Crude peptide was precipitated from cold ether and collected by filtration. Yield 33.0 gram (Synthesis yield 194.2%). 17 g crude peptide was purified by reverse-phase HPLC; collected fractions with purity over 90% were pooled and lyophilized. Yield final product 9.25 g (purification yield 54.4%).

Preparation of (MAA-PEO₄-VN): MAA-PEO₄-Lys-Gly-Gly-Pro-Gln-Val-Thr-Arg-Gly-Asp-Val-Phe-Thr-Met-Pro-NH₂ (SEQ ID NO:27): The peptide was synthesized on 1 mmol Fmoc-Rink Amide resin via Fmoc chemistry. Protecting groups used for amino acids are: t-Butyl group for and Asp and Thr, Trt group for Gln, Pbf for Arg, Boc for Lys. Fmoc protected amino acids were purchased from EMD Biosciences; Fmoc-PEG₄-OH was purchased from Quanta Biodesign. Reagents for coupling and cleavage were purchased from Aldrich. Solvents were purchased from Fisher Scientific. The peptide chain was assembled on resin by repetitive removal of the Fmoc protecting group and coupling of protected amino acid. HBTU and HOBt were used as coupling reagents and NMM was used as base. 20% piperidine in DMF was used as de-Fmoc-reagent. Methacrylic acid (MAA) was coupled to the amino group of PEG₄ after removal of the Fmoc protecting group. After the last coupling, resin was treated with TFA/TIS/H2O (95:3:2, v/v/v) for cleavage and removal of the side chain protecting groups. Crude peptide was precipitated from cold ether and collected by filtration. Yield 4.0 gram (Synthesis yield 210.1%). Crude peptide was purified by reverse-phase HPLC; collected fractions with purity over 90% were pooled and lyophilized. Yield final product 1.035 g (purification yield 25.9%).

The products were provided by American Peptide in >90% purity and were used without further purification. Ethanol was used as non-reactive diluent in the process and was purchased from Sigma-Aldrich.

### General Procedure for the Preparation of Functionalized Peptide Polymer Formulations:

Into a separate 10 ml scintillation vial quantities of 400 μL of 2-hydroxyethyl methacrylate was added, subsequently ethanol was added along with 40 μL of tetra(ethylene glycol) dimethacrylate, 15 μL of Darocur 1173 (10% in ethanol), 50 μL Irgacure 819 (1% in ethanol) and 9.5 ml of ethanol. This recipe amounts to a 1% formulation in ethanol. For libraries involving glycerol monomethacrylate isomers and glycerol 1,3-diglycerol dimethacrylate the same volume of 400 μL and 40 μL were used respectively. Ac-Lys(MAA)-Gly-Gly-Asn-Gly-Glu-Pro-Arg-Gly-Asp-Thr-Tyr-Arg-Ala-Tyr-NH₂ (MAA-SEQ ID NO:19), Ac-Lys(MAA)-Gly-Gly-Pro-Gln-Val-Thr-Arg-Gly-Asp-Val-Phe-Thr-Met-Pro-NH₂ (MAA-SEQ ID NO:28), were added to create a peptide gradient of 8.74 mg, 6.55 mg, 4.37 mg, 2.19 mg and 0.87 mg per 10 ml of monomer formulation described above in table 2, 3 and 7. While MAA-PEG₄-Lys-Gly-Gly-Pro-Gln-Val-Thr-Arg-Gly-Asp-Val-Phe-Thr-Met-Pro-NH₂ (MAA-PEG₄-SEQ ID NO:28) were added in a 4.37 mg, 3.28 mg, 2.19 mg, 1.09 mg and 0.437 mg to respective formulations shown in table 4 and 5. The Ac-Lys(MAA)-Gly-Gly-Asn-Gly-Glu-Pro-Arg-Gly-Asp-Thr-Tyr-Arg-Ala-Tyr-NH₂-MAA (MAA-SEQ ID NO:19) was added in 2.0 mg, 1.5 mg, 1.0 mg and 0.5 mg but can be increased to as much as 8.0 mg as seen in Table 6. The notations PA, PB, PC, PD, PE and PF refer to formulations mixed with peptide. The number refers to the peptide gradient created to test cell response.

### General Procedure for the Preparation of Cyclic Functionalized Peptide Polymer Formulations:

Into a 10 ml scintillation vial, quantities of 80 μL of Glycerol Monomethacrylate along with 8 μL of Glycerol 1,3-Diglycerolate Dimethacrylate were added. Subsequently, 15 μL of Darocur 1173 (10% in ethanol), 50 μL Irgacure 819 (1% in ethanol) and 9.92 ml of ethanol as a solvent were also added. This recipe amounts to a 1% formulation in ethanol. The formulation was set aside for coating into 6-wps.

Another hydrogel formulation, a HEMA/TEGDMA/Cyclic peptide surface was prepared for screening which contained 80 μL of 2-hydroxyethyl Methacrylate and 30 μL Tetra(ethylene glycol) Dimethacrylate. 30 μl, of Darocur 1173 (10% in ethanol), 20 μL Irgacure 819 (1% in ethanol) and 9.92 ml of ethanol as a solvent. 1 mg of functionalized cyclic peptide (c(RGDyK-MAA) (SEQ ID NO: 34)) was dissolved in 40 μL of 18 Mega OHM water in 10 μL increments. 960 μL of the acrylate hydrogel was added to the dissolved peptide for a total of 1 ml of a 1 mM peptide concentration solution.

Into 3 ml scintillation vials, either 1 mg of functionalized cyclic peptide, c(RGDyK-MAA) (SEQ ID NO: 34) or 1.7 mg of functionalized cyclic peptide, c(RGDyK-MAA) (SEQ ID NO: 34) was used. To the dry peptide, 80 μL of 18 Mega OHM water in 20 μL increments was added until the peptide was dissolved. To the dissolved peptide, 1920 μl of the acrylate hydrogel (either the glycerol monomethacrylate+1,3-diglycerolate dimethacrylate or HEMA+TEGDMA) was added for a total of 2 ml of a 0.5 mM peptide concentration solution.

### General Procedure for Coating of Peptide-Polymer Formulations in 6 Well Plates:

Cellbind® (Corning Incorporated, Corning, N.Y.) treated polystyrene six-well plates (6-wp) were removed from packaging and placed in large nitrogen purge box which was continuously being purged with nitrogen gas. The humidity level in the purge box was less than 30% before dispensing formulations. A semi-automated pippettor was used to dispense 26 μL into each well. After the formulation spread and over the well surfaces, the solvent ethanol was removed by in vacuum oven at 25 to 30 in Hg for 5 minutes before curing.

### Procedure for UV Curing Peptide-Polymer Coating

A “Xenon Model RC-801 high intensity pulsed Ultraviolet (UV) light curing system” from INPRO Technologies, Inc. was used in curing. The plates were constantly being purged with nitrogen in order to create an inert environment (for the coatings) during curing. The cure time was set (i.e. 60 sec. in this study).

### Procedure for Washing of Peptide-Polymer Coated Plates:

After UV curing of peptide-acrylate coatings, 1-5 ml of ethanol was dispensed into wells of plates and placed on rocker to agitate for 30-60 minutes in order to remove any residual monomers. The 6 well plates were rinsed with 18 Mega OHM water five times, then dried overnight and submitted for cell testing.

### Procedure for Culturing Human Embryonic Stem Cells:

H7 hES cells were provided as part of collaboration agreement with Geron Corporation and cultured according to their protocols. Briefly, cells were cultured on MG-coated TCT flasks in chemically defined medium (X-Vivo-10, 80 ng/ml hbFGF, 5 ng/ml hTGF-β1). Cells were passaged every 4-5 days at the seeding density of 10×10⁶ cells/T75 flask using Geron's sub-cultivation procedure. For the experiments, cells were seeded in 6-well plates at the density of 1×10⁶/well in chemically defined media. Cell morphology was observed daily. Cells were stained with crystal violet on day 4 for visual assessment of cell number, colony morphology, and distribution.

Procedure for Culturing Neuronal Progenitor Stem Cells: ReNcell VM cells from Millipore (Temecula, Calif.) were routinely expanded on Laminin coated T75 cm2 tissue culture flasks (Corning, N.Y.) in ReNcell NSC Maintenance Medium (Millipore, Temecula, Calif.) containing 20 ng/mL FGF-2 and 20 ng/mL EGF (Millipore, Temecula, Calif.). For maintenance and growth of undifferentiated cells, the medium was changed every other day. All cells in culture were maintained at 37° C. in a humidified atmosphere of 95% air/5% CO₂. Cells were passaged once a week using Accutase (Millipore, Temecula, Calif.).

Undifferentiated neural progenitors stem cells were seeded in 6 wells microplate at 150000 cells per well in ReNcell NSC Maintenance Medium containing 20 ng/mL FGF-2 and 20 ng/mL EGF. 24 h after seeding cellular attachment and cell morphology was assessed using Ziess Axiovert 200M inverted microscope. Cells were grown on each surface for 4 days. At day 2 and day 3 after seeding, cells were detached from the surface by Accutase treatment and counted. Doubling time was calculated in the exponential phase of the growth curve (previously estimated between day 2 and 3 after seeding). Laminin coated TCT microplates were used as positive control for neural progenitor stem cells growth. Neural progenitors stem cells were seeded in ReNcell NSC Maintenance Medium containing 20 ng/mL FGF-2 and 20 ng/mL EGF in 6 wells microplate at 250000 cells per well and were cultured at 37° C. under air/5% CO2. The next day, differentiation was initiated by removing the medium from each well and replacing with fresh ReNcell NSC Maintenance Medium that does not contain FGF-2 and EGF. The medium was replaced with fresh ReNcell NSC Maintenance Medium every 2-3 days for 10 days.

FIGS. 4 and 5 show photomicrographs of H7 human embryonic stem cells cultured on control surfaces Matrigel™ and Synthemax™ compared to embodiments of the peptide-polymer cell culture surfaces of the present invention. Peptide-polymer showed human stem cell growth and morphology approaching that seen on control surfaces after four days in culture.

FIGS. 6A-H show photomicrographs of neural progenitor cells, after one day in culture (FIGS. 6 A-D) and after three days in culture (FIGS. 6E-H) growing on Laminin™ surface from Corning, Incorporated (FIGS. 6A and E), on a cyclic functionalized RGD peptide surface made with HEMA and TEGDMA (FIGS. 6B and F), on a cyclic functionalized RGD peptide (cyclic SEQ ID NO: 34) surface made with glycerol methacrylate and 1,3-diglycerolate dimethacrylate (FIGS. 6C and G) and on a Synthemax™ surface from Corning Incorporated (FIGS. 6D and H).

Thus, embodiments of PEPTIDE POLYMER CELL CULTURE ARTICLES AND METHODS OF MAKING are disclosed. One skilled in the art will appreciate that the arrays, compositions, kits articles and methods described herein can be practiced with embodiments other than those disclosed. The disclosed embodiments are presented for purposes of illustration and not limitation.

What is claimed is:

1. A functionalized peptide monomer wherein the functionalized peptide monomer is described by the formula: Aₘ-S_{y}-Xaaₙ-S_{y1}-Z-S_{y2}Aₘ₁ where A is a polymerization moiety, m is an integer from 1 to 6, m1 is an integer from 1 to 6, Xaaₙ is independently any amino acid, n is an integer from 0 to 6, S is a spacer having the formula (O—CH₂CHR′)ₘ₂ where R′ is H or CH₃ and m2 is an integer from 0 to 20, y is an integer from 0 to 30, y1 is an integer from 0 to 30, y2 is an integer from 0 to 30, and Z is a cell adhesive peptide.

2. The functionalized peptide monomer of claim 1 wherein the cell adhesive peptide (Z) comprises the sequence: KGGGQKCIVQTTSWSQCSKS (SEQ ID NO:1); GGGQKCIVQTTSWSQCSKS(SEQ ID NO:2); KYGLALERKDHSG (SEQ ID NO:3); YGLALERKDHSG (SEQ ID NO:4); KGGSINNNRWHSIYITRFGNMGS (SEQ ID NO:5); GGSINNNRWHSIYITRFGNMGS (SEQ ID NO:6); KGGTWYKIAFQRNRK (SEQ ID NO:7); GGTWYKIAFQRNRK (SEQ ID NO:8); KGGTSIKIRGTYSER (SEQ ID NO:9); GGTSIKIRGTYSER (SEQ ID NO:10); KYGTDIRVTLNRLNTF (SEQ ID NO:11); YGTDIRVTLNRLNTF (SEQ ID NO:12); KYGSETTVKYIFRLHE (SEQ ID NO:13); YGSETTVKYIFRLHE (SEQ ID NO:14); KYGKAFDITYVRLKF (SEQ ID NO:15); YGKAFDITYVRLKF (SEQ ID NO:16); KYGAASIKVAVSADR (SEQ ID NO:17); YGAASIKVAVSADR(SEQ ID NO:18); KGGNGEPRGDTYRAY(SEQ ID NO:19); GGNGEPRGDTYRAY (SEQ ID NO:20) CGGNGEPRGDTYRAY (SEQ ID NO:21); GGNGEPRGDTRAY (SEQ ID NO:22); KYGRKRLQVQLSIRT (SEQ ID NO:23); YGRKRLQVQLSIRT (SEQ ID NO:24); KGGRNIAEIIKDI (SEQ ID NO:25); GGRNIAEIIKDI (SEQ ID NO:26); KGGPQVTRGDVFTMP (SEQ ID NO:27); GGPQVTRGDVFTMP (SEQ ID NO:28); GGPQVTRGDVFTMPK (SEQ ID NO:29); GRGDSPK (SEQ ID NO:30); KGGAVTGRGDSPASS(SEQ ID NO:31); GGAVTGRGDSPASS (SEQ ID NO:32); Yaa₁PQVTRGNVFTMP (SEQ ID NO:32); RGDYK (SEQ ID NO:34), or combinations.

3. The functionalized peptide monomer of claim 1 wherein the cell adhesive peptide comprises KGGPQVTRGDVFTMP (SEQ ID NO:27) or GGPQVTRGDVFTMP (SEQ ID NO:28).

4. The functionalized peptide of claim 3 wherein the polymerization moiety A comprises an acrylate or a methacrylate moiety.

5. The functionalized peptide of claim 4 wherein the polymerization moiety is methacrylic acid.

6. The functionalized peptide of claim 1 wherein the spacer is a polyethylene oxide with 20 or fewer repeating units.

7. The functionalized peptide of claim 1 wherein the spacer is PEG₄.

8. A polymer formed from a mixture comprising the functionalized peptide of claim 1 and at least one photopolymerizable monomer.

9. The polymer of claim 8 wherein the at least one photopolymerizable monomer is HEMA, TEGDMA, glycerol monomethacrylate or glycerol 1,3-diglycerolate dimethacrylate.

10. The polymer of claim 8 wherein the polymer is formed from a mixture comprising a functionalized peptide, HEMA and TEGDA.

11. The polymer of claim 8 wherein the polymer is formed from a mixture comprising a functionalized peptide, Glycerol monomethacrylate and glycerol 1,3-diglycerolate dimethacrylate.

12. The polymer of claim 8 wherein the polymer is formed in situ.

13. A method of making a cell culture surface comprising the steps of:
providing a mixture of functionalized peptide monomer and at least one
(meth)acrylate monomer;
applying the mixture to a cell culture substrate;
polymerizing the monomers in situ to form a peptide-polymer.

14. The method of claim 13 wherein the (meth)acrylate monomer comprises HEMA, TEGDA, Glycerol monomethacrylate or glycerol 1,3-diglycerolate dimethacrylate or combinations.

15. The method of claim 13 wherein the methacrylate monomer comprises a combination of HEMA and TEGDA.

16. The method of claim 8 wherein the functionalized peptide monomer is described by the formula: Aₘ-S_{y}-Xaaₙ-S_{y1}-Z-S_{y2}-Aₘ₁ where A is a polymerization moiety, m is an integer from 1 to 6, m1 is an integer from 1 to 6, Xaaₙ is independently any amino acid, n is an integer from 0 to 6, S is a spacer, y is an integer from 0 to 30, y1 is an integer from 0 to 30, y2 is an integer from 0 to 30, and Z is a cell adhesive peptide.

17. The method of claim 16 wherein Xaa is Lys and n=1.

18. The method of claim 16 wherein A is an acrylate or methacrylate.

19. The method of claim 16 wherein S is PEO and m=4.

20. The method of claim 16 wherein Z is a cell adhesive peptide selected from the group consisting of: KGGGQKCIVQTTSWSQCSKS (SEQ ID NO:1); GGGQKCIVQTTSWSQCSKS(SEQ ID NO:2); KYGLALERKDHSG (SEQ ID NO:3); YGLALERKDHSG (SEQ ID NO:4); KGGSINNNRWHSIYITRFGNMGS (SEQ ID NO:5); GGSINNNRWHSIYITRFGNMGS (SEQ ID NO:6); KGGTWYKIAFQRNRK (SEQ NO:7); GGTWYKIAFQRNRK (SEQ ID NO:8); KGGTSIKIRGTYSER (SEQ ID NO:9); GGTSIKIRGTYSER (SEQ ID NO:10); KYGTDIRVTLNRLNTF (SEQ NO:11); YGTDIRVTLNRLNTF (SEQ ID NO:12); KYGSETTVKYIFRLHE (SEQ ID NO:13); YGSETTVKYIFRLHE (SEQ ID NO:14); KYGKAFDITYVRLKF (SEQ NO:15); YGKAFDITYVRLKF (SEQ ID NO:16); KYGAASIKVAVSADR (SEQ NO:17); YGAASIKVAVSADR(SEQ ID NO:18); KGGNGEPRGDTYRAY(SEQ NO:19); GGNGEPRGDTYRAY (SEQ ID NO:20) CGGNGEPRGDTYRAY (SEQ ID NO:21); GGNGEPRGDTRAY (SEQ ID NO:22); KYGRKRLQVQLSIRT (SEQ ID NO:23); YGRKRLQVQLSIRT (SEQ ID NO:24); KGGRNIAEIIKDI (SEQ NO:25); GGRNIAEIIKDI (SEQ ID NO:26); KGGPQVTRGDVFTMP (SEQ ID NO:27); GGPQVTRGDVFTMP (SEQ ID NO:28); GGPQVTRGDVFTMPK (SEQ ID NO:29); GRGDSPK (SEQ NO:30); KGGAVTGRGDSPASS(SEQ NO:31); GGAVTGRGDSPASS (SEQ NO:32); Yaa₁PQVTRGNVFTMP (SEQ ID NO:32); RGDYK (SEQ ID NO:34), where the peptide sequences may be linear or cyclic, or combinations.

21. A cell culture surface made by the method of claim 13.

22. A functionalized cyclic peptide monomer wherein the functionalized peptide monomer is described by the formula: Z_{c}-S_{y}-Xaaₙ-Aₘ wherein Zc is a cell adhesive cyclic peptide, S is a spacer, y is an integer from 0 to 30, Xaaₙ is independently any amino acid, n is an integer from 0 to 6, A is a polymerization moiety and m is an integer from 1 to 6.

23. The functionalized cyclic peptide monomer of claim 22 wherein the polymerization moiety A comprises an acrylate or a methacrylate moiety.

24. The functionalized cyclic peptide of claim 22 wherein the polymerization moiety is methacrylic acid.

25. The functionalized cyclic peptide of claim 22 wherein the spacer is a polyethylene oxide with 20 or fewer repeating units.

26. The functionalized cyclic peptide of claim 22 wherein the spacer is PEG₄.

27. A polymer formed from a mixture comprising the functionalized cyclic peptide of claim 22 and at least one photopolymerizable monomer.

28. The polymer of claim 27 wherein the at least one photopolymerizable monomer is HEMA, TEGDMA, glycerol monomethacrylate or glycerol 1,3-diglycerolate dimethacrylate.

29. The polymer of claim 27 wherein the polymer is formed from a mixture comprising a functionalized peptide, HEMA and TEGDA.

30. The polymer of claim 27 wherein the polymer is formed from a mixture comprising a functionalized peptide, Glycerol monomethacrylate and glycerol 1,3-diglycerolate dimethacrylate.
